(19) United States
(12) Patent Application Publication
(10) Pub. No.: US 2018/0164333 A1
(43) Pub. Date: Jun. 14, 2018

(54) TEST PLATE PROCESSOR DEVICE AND METHODS FOR USING SAME

(71) Applicant: OBOTICS, LLC, LOS ANGELES, CA (US)

(72) Inventor: Lane YAGO, Walnut Creek, CA (US)

(73) Assignee: OBOTICS, LLC, LOS ANGELES, CA (US)

(21) Appl. No.: 15/839,681

(22) Filed: Dec. 12, 2017

Related U.S. Application Data
(60) Provisional application No. 62/433,667, filed on Dec. 13, 2016.

(51) Int. Cl. G01N 35/00; C12M 1/22; C12M 1/00

(52) U.S. Cl. CPC G01N 35/00029; G01N 35/00871; G01N 2035/00099; C12M 23/10; C12M 23/38; G01N 35/0099

(57) ABSTRACT

A test plate processing system for processing multiple test plates, comprising one or more software controlled processing devices, a liquid handler configured to place a predetermined amount of a test substance on the plate when the cover is in the first position, a software controlled test plate storage device having one or more test plate storage modules configured to house a plurality of test plates, and a software controlled test plate transport device configured to move one or more test plates to and from the test plate storage device. Each of the processing devices can have a working surface and a lifting mechanism configured to move a cover of the test plate between a first position and a second position relative to a plating surface of the test plate, wherein the cover is further away from the plating surface in the first position than in the second position.

### PRIORITY CLAIM

The present application claims priority from U.S. Patent Application No. 62/433,667, filed on Dec. 13, 2016, titled TEST PLATE PROCESSOR DEVICE AND METHODS FOR USING SAME, the content of which is incorporated by reference herein in its entirety. The benefit of priority is claimed under the appropriate legal basis including, without limitation, under 35 U.S.C. § 119(e).

### BACKGROUND OF THE DISCLOSURE

### Field of the Disclosure

The disclosure relates to automated devices, in particular, automated laboratory or testing devices that can be used to automate the processing of test samples used in fields such as, but not limited to, microbiology.

### Description of the Related Art

The automated testing equipment embodiments disclosed herein can be used in a variety of fields, including without limitation the fields of chemistry, biology, and/or microbiology. But there are many other fields in which the embodiments disclosed herein may be used and may be useful. In microbiology, for example, one may need to grow cultures of the micro-organisms being studied. Historically, this has been accomplished in small round containers known as petri dishes. In such culture processing, growth media (often a gel-like liquid known as “agar”) can be put into the petri dish, and a small sample of the micro-organism or micro-organisms can be placed in the growth media. A lid can be used to cover the petri dish before the petri is placed in an incubator to allow the micro-organisms to grow. Subsequent measurements or testing procedures can then be performed on the cultured sample.

3M™ has developed a small laboratory testing card called a PETRIFILM™ Plate. The PETRIFILM Plate can come pre-prepared with a water-soluble gelling agent, nutrients, and indicator, all of the components that may be needed for microbial growth with no agar preparation required. Instead of a lid (as on a petri dish), the PETRIFILM Plate can be covered with a thin film cover that is attached at one end of the plate, and forms part of the PETRIFILM Plate, so that the cover can be lifted up while still remaining attached to the plate.

One example of how the PETRIFILM Plate can be used in conventional practice is now described. Although steps are described in a particular order, other steps may be performed, one or more of the steps described below may be omitted, or one or more of the steps may be performed in a different order. First, holding the PETRIFILM Plate, the cover of the PETRIFILM Plate can be lifted to expose the plating surface of the PETRIFILM Plate. Micro-organisms, which can be in a solution, can then be dispensed directly onto the plating surface. A pipet can be used to dispense the solution. The film can be lowered to cover the micro-organism. Then, using a small flat tool, the dispensed amount of micro-organisms or test solution can be spread out by gently pressing on the film. The PETRIFILM Plate can then be placed in an incubator. This has typically been a manual process, with no known processes that can effectively automate the use of the PETRIFILM Plate for laboratory testing or otherwise. Because a typical test procedure can require the processing of many PETRIFILM Plates, a need exists for devices and methods for automating the processing of the PETRIFILM Plates.

### SUMMARY

The systems, methods and devices described herein have innovative aspects, no single one of which is indispensable or solely responsible for their desirable attributes. Without limiting the scope of the claims, some of the advantageous features will now be summarized.

Some embodiments disclosed herein relate to automated processing devices for testing of substances, which can include micro-organisms, using Petri dishes, films or plates. Some embodiments are directed to a test plate processing device, which can have a housing member configured to support a test plate having a cover, a suction member or suction element configured to couple with the cover of the test plate, and a support member configured to move the suction member between a first position and a second position. In the first position, the suction member can be positioned away from a plating surface of the test plate. In the second position, the suction member is positioned adjacent to the plating surface of the test plate.

Some embodiments are directed to a method of processing a culture test plate using an automated processing device, comprising supporting the test plate having a cover with the processing device, moving the cover away from a plating surface of the test plate, dispensing a desired amount of a test substance on the plating surface of the test plate, and moving the cover against the plating surface of the test plate.

There can be significant advantages to using any of the embodiments of the automated processing device for processing test plates disclosed herein. First, using an automated processing device can eliminate manual processing of PETRIFILM Plates, which can increase throughput volume, improve consistency, precision, and accuracy of testing, and eliminate potential Repetitive Stress Syndrome for technicians, as well as other ailments. Additionally, the embodiments of the automated processing device disclosed herein can allow for integration with automated liquid handlers and robotic grippers.

Any of the embodiments disclosed herein can have any combination of any of the components, details, or other features of any of the following arrangements.

Arrangement 1: A processing device for processing one or more test plates, comprising:
a housing;
a base member coupled with the housing, the base member having a working surface against which a test plate is operably positionable,
a support member in communication with the housing and movable between a first position and a second position; and
a suction element supported directly or indirectly by the support member;
wherein:

the processing device can communicate a suction force through the suction element;

the processing device can be configured such that, when the support member is in the first position, an end portion of the suction element can be positioned away from a working surface of the base member by a first distance, the first distance being in a direction that is normal to the working surface of the base member;

the processing device can be configured such that, when the support member is in the second position, the end portion of the suction element can be positioned within a second distance away from the working surface of the base member, the second distance being in a direction that is normal to the working surface of the base member; and

the second distance can be significantly smaller than the first distance.

Arrangement 2: The processing device of Arrangement 1, further comprising at least one opening that can extend through the working surface of the base member, wherein the processing device can communicate a suction force through the at least one opening;

Arrangement 3: The processing device of any one of the previous Arrangements, comprising an air passageway that can extend through at least a portion of the housing. The air passageway can be in communication with the at least one opening that can extend through the working surface of the base member.

Arrangement 4: A test plate processing system, comprising the processing device of any one of the previous Arrangements and a source of reduced pressure that can provide a reduced pressure to at least one of the suction elements and the opening that can extend through the base member.

Arrangement 5: The processing device of any one of the previous Arrangements, wherein the processing device can be configured such that, when the support member is in the second position, the end portion of the suction element can be positionable so as to be in contact with the working surface of the base member.

Arrangement 6: The processing device of any one of the previous Arrangements, wherein the processing device can be configured such that, when the support member is in the first position, the end portion of the suction element can be positioned to a side of the base member so that the suction element is not positioned directly over the base member.

Arrangement 7: The processing device of any one of the previous Arrangements, wherein the first distance can be at least approximately one-half inch.

Arrangement 8: The processing device of any one of the previous Arrangements, wherein the first distance can be at least approximately three-quarters of an inch.

Arrangement 9: The processing device of any one of the previous Arrangements, wherein the first distance can be at least 20 times greater than a thickness of a test plate that is positionable on the processing device.

Arrangement 10: The processing device of any one of the previous Arrangements, wherein the first distance can be at least twice as great as a thickness of a test plate holder that is positionable on the processing device.

Arrangement 11: The processing device of any one of the previous Arrangements, comprising an actuator that can controllably move the support member between the first and second positions.

Arrangement 12: The processing device of any one of the previous Arrangements, wherein the support member can be coupled to a shaft of an actuator rotatable between a first angular orientation wherein the support member is in the first position and a second angular orientation wherein the support member is in the second position.

Arrangement 13: The processing device of any one of the previous Arrangements, wherein a first end portion of the support member can be coupled with a rotatable shaft and the suction element can be coupled with a second end portion of the support member.

Arrangement 14: The processing device of any one of the previous Arrangements, wherein the suction element can comprise a suction cup.

Arrangement 15: The processing device of any one of the previous Arrangements, comprising a limit switch that can stop the support member when the support member has moved to the first position.

Arrangement 16: The processing device of any one of the previous Arrangements, wherein the suction element can rotate about an arcuate path between the first and second positions.

Arrangement 17: The processing device of any one of the previous Arrangements, comprising a plurality of openings that can extend through the working surface of the base member.

Arrangement 18: The processing device of any one of the previous Arrangements, wherein the test plate is a 3M PETRIFILM plate.

Arrangement 19: The processing device of any one of the previous Arrangements, further comprising a holder element for supporting the test plate during processing, the holder element being operably coupleable with the base member of the housing.

Arrangement 20: A test plate processing kit, comprising the processing device of any one of the previous Arrangements and a test plate.

Arrangement 21: A test plate processing system, comprising the processing device of any one of the previous Arrangements and a source of reduced pressure that can provide a reduced pressure to at least one of the suction elements.

Arrangement 22: A test plate processing system, comprising:
one or more processing devices of any one of the previous Arrangements;
an automated test plate storage device having one or more test plate storage modules that can house a plurality of test plates; and
a test plate transport device that can move one or more test plates to and from the test plate storage device, the test plate storage device being controllable using a control system.

Arrangement 23: The test plate processing system of any one of the previous Arrangements, comprising two or more processing devices.

Arrangement 24: The test plate processing system of any one of the previous Arrangements, further comprising a multi-plate holder that can support a first test plate on a first portion of the multi-plate holder, a second plate on a section portion of the multi-plate holder, and a third plate in a third portion of the multi-plate holder.

Arrangement 25: The test plate processing system of any one of the previous Arrangements, further comprising a spreader device that can spread a test substance on a test plate, the spreader device being controllable using a control system.

Arrangement 26: The test plate processing system of any one of the previous Arrangements, wherein the spreader device can comprise two or more selectable spreader elements wherein the spreader device can be controlled by a control system that can selectively position a desired spreader element of the two or more selectable spreader elements in an operable position.

Arrangement 27: The test plate processing system of any one of the previous Arrangements, further comprising a robotic arm that can move one or more test plates from one location in the test plate processing system to a second location in the test plate processing system.

Arrangement 28: The test plate processing system of any one of the previous Arrangements, wherein the test plate transport device can comprise one or more suction elements that can selectively apply a suction force against a test plate when the one or more suction elements are in contact with the test plate.

Arrangement 29: A test plate processing system configured to process multiple test plates, comprising:
one or more software controlled processing devices, each having a working surface, and a lifting mechanism that can move a cover of the test plate between a first position and a second position relative to a plating surface of the test plate, wherein:

the plating surface is a surface of the test plate selectively coverable with a cover of the test plate; and the cover can be further away from the plating surface in the first position than in the second position;
a liquid handler that can place a predetermined amount of a test substance on the plating surface of the test plate when the cover is in the first position;
a software controlled test plate storage device having one or more test plate storage modules that can house a plurality of test plates; and
a software controlled test plate transport device that can move one or more test plates to and from the test plate storage device.

Arrangement 30: The test plate processing system of Arrangement 29, wherein the lifting mechanism of the one or more processing devices can comprise a suction element.

Arrangement 31: The test plate processing system of any one of Arrangements 29-30, comprising two or more processing devices.

Arrangement 32: The test plate processing system of any one of Arrangements 29-31, comprising three or more processing devices and a multi-plate holder having a first portion that can support a first test plate thereon, a second portion that can support a second test plate thereon; and a third portion that can support a third test plate thereon.

Arrangement 33: The test plate processing system of any one of Arrangements 29-32, comprising a source of suction that can provide suction to at least one of an opening formed in the working surface of the one or more processing devices and the lifting mechanism.

Arrangement 34: The test plate processing system of any one of Arrangements 29-33, further comprising a spreader device that can spread a test substance on a test plate, the spreader device being controllable using a control system.

Arrangement 35: The test plate processing system of any one of Arrangements 29-34, wherein the spreader device can comprise two or more selectable spreader elements wherein the spreader device can be controlled by a control system that can selectively position a desired spreader element of the two or more selectable spreader elements in an operable position.

Arrangement 36: The test plate processing system of any one of Arrangements 29-35, further comprising a robotic arm that can move one or more test plates from one location in the test plate processing system to a second location in the test plate processing system.

Arrangement 37: The test plate processing system of any one of Arrangements 29-36, wherein the test plate transport device can comprise one or more suction elements that can selectively apply a suction force against a test plate when the one or more suction elements are in contact with the test plate.

Arrangement 38: A method of positioning a substance on a plating surface of a test plate having a cover using a software controlled automated processing system, comprising:
positioning the test plate in an operable position on a processing device;
selectively coupling a portion of the processing device with a portion of the cover of the test plate;
moving the cover away from the plating surface of the test plate;
dispensing a desired amount of a test substance on the plating surface of the test plate; and
moving the cover against the plating surface of the test plate.

Arrangement 39: The method of processing a test plate of Arrangement 38, further comprising moving a test plate directly or indirectly from a storage device to the processing device.

Arrangement 40: The method of processing a test plate of any one of Arrangements 38-39, further comprising moving a test plate directly or indirectly from the processing device to the storage device.

Arrangement 41: The method of processing a test plate of any one of Arrangements 38-40, further comprising moving one or more test plates to an incubator using software controlled automated devices.

Arrangement 42: The method of processing a test plate of any one of Arrangements 38-41, comprising applying suction to selectively couple the cover to a support arm of the processing device.

Arrangement 43: The method of processing a test plate of any one of Arrangements 38-42, wherein supporting the test plate with the processing device can comprise applying suction to a bottom surface of the test plate.

Arrangement 44: The method of processing a test plate of any one of Arrangements 38-43, further comprising storing 15 or more test plates in a storage module.

Arrangement 45: The method of processing a test plate of any one of Arrangements 38-44, further comprising moving a storage module having 15 or more test plates supported thereby to an incubator.

Arrangement 46: A test plate processing device substantially as hereinbefore described with reference to any one of the embodiments or to any one of the accompanying drawings disclosed herein.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through the use of the accompanying drawings.

Embodiments of the present disclosure will now be described hereinafter, by way of example only, with reference to the accompanying drawings in which:

FIG. 1 is an isometric view of an embodiment of a test plate processing device, showing the processing device in a first or open state or position.

FIG. 2 is an isometric view of the embodiment of the processing device shown in FIG. 1, showing the processing device in a second or closed state or position.

FIG. 3 is a top view of the embodiment of the processing device shown in FIG. 1, showing the processing device in the first state or position.

FIG. 4 is a top view of the embodiment of the processing device shown in FIG. 1, showing the processing device in the second state or position.

FIG. 5 is a back view of the embodiment of the processing device shown in FIG. 1, showing the processing device in the first state or position.

FIG. 6 is a back view of the embodiment of the processing device shown in FIG. 1, showing the processing device in the second state or position.

FIG. 7 is a side view of view of the embodiment of the processing device shown in FIG. 1, showing the processing device in the first state or position.

FIG. 8 is a side view of the embodiment of the processing device shown in FIG. 1, showing the processing device in the second state or position.

FIG. 9 is an isometric view of the embodiment of the processing device shown in FIG. 1, showing an embodiment of a test plate support member positioned on the embodiment of the housing member, and with the processing device in the first state or position.

FIG. 10 is an isometric view of the embodiment of the processing device shown in FIG. 1, showing the embodiment of the test plate support member shown in FIG. 9 positioned on the embodiment of the housing member, and with the processing device in the second state or position.

FIG. 11 is an isometric view of the embodiment of the processing device shown in FIG. 1, showing an embodiment of a test plate support member positioned on the embodiment of the housing member, the suction member in contact with the cover of the testing plate, and with the processing device in the first state or position.

FIG. 12 is an exploded view of the embodiment of the processing device shown in FIG. 1.

FIG. 13 is a top view of the embodiment of the base plate or base member of the embodiment of the processing device shown in FIG. 1.

FIG. 14 is an isometric view of the embodiment of the housing member component of the embodiment of the processing device shown in FIG. 1.

FIG. 15 is another isometric view of the embodiment of the housing member component shown in FIG. 15.

FIG. 16 is an isometric view of the embodiment of the test plate holder shown in FIG. 9.

FIG. 17 is an isometric view of another embodiment of a test plate holder.

FIG. 18 is an isometric view of the embodiment of the test plate holder shown in FIG. 17, showing an underside of the holder.

FIG. 19 is an isometric view of an embodiment of multi-plate holder.

FIG. 20 is an isometric view of the embodiment of the multi-plate holder shown in FIG. 19, showing an underside of the multi-plate holder.

FIG. 21 is an isometric view of the embodiment of the multi-plate holder of FIG. 19 positioned on a series of processing devices.

FIG. 22 is an isometric view of another embodiment of a test plate processing device, showing the processing device in a first or open state or position.

FIG. 23 is an isometric view of the embodiment of the processing device shown in FIG. 22, showing the processing device in the first or open state or position.

FIG. 24 is an isometric view of an embodiment of a processing system.

FIG. 25 is a top view of the embodiment of the processing system shown in FIG. 24.

FIG. 26 is an isometric view of the embodiment of the test plate storage system shown in FIG. 24.

FIG. 27 is a top view of the embodiment of the test plate storage system shown in FIG. 24.

FIG. 28 is an enlarged view of a portion of the embodiment of the test plate storage system shown in FIG. 26.

FIG. 29 is an enlarged view of a portion of the embodiment of the test plate processing system shown in FIG. 24.

FIG. 30 is an enlarged view of a portion of the embodiment of the test plate processing system shown in FIG. 24, showing the embodiment of the spreader device of the processing system embodiment shown in FIG. 24.

FIG. 31 is another enlarged view of the embodiment of the spreader device shown in FIG. 31.

FIG. 32 is another enlarged view of the embodiment of the spreader device shown in FIG. 31.

FIG. 33 is an enlarged view of a portion of the embodiment of the test plate processing system shown in FIG. 24, showing the embodiment of the test plate transport device of the processing system embodiment shown in FIG. 24.

FIG. 34 is an enlarged view of a portion of the embodiment of the test plate processing system shown in FIG. 24, showing the embodiment of the test plate transport device lifting a test plate.

FIG. 35 is an enlarged view of a portion of the embodiment of the test plate processing system shown in FIG. 24, showing the embodiment of the test plate transport device moving a test plate.

FIG. 36 is an enlarged view of a portion of the embodiment of the test plate processing system shown in FIG. 24, showing the embodiment of the test plate transport device depositing a test plate on a predetermined portion of the multi-plate holder embodiment shown in FIG. 24.

FIG. 37 is an enlarged view of a portion of the embodiment of the test plate processing system shown in FIG. 24, showing the robotic arm engaging the multi-plate holder.

FIG. 38 is an enlarged view of a portion of the embodiment of the test plate processing system shown in FIG. 24, showing the robotic arm depositing the multi-plate holder on the array of processing devices.

FIG. 39 is an enlarged view of a portion of the embodiment of the test plate processing system shown in FIG. 24, showing the robotic arm engaging the multi-plate holder that is positioned on the loading nest.

FIG. 40 is an enlarged view of a portion of the embodiment of the test plate processing system shown in FIG. 24, showing the robotic arm depositing the multi-plate holder on the positioning device.

FIG. 41 is an isometric view of an embodiment of a stirring device.

FIG. 42 is a top view of the embodiment of the stirring device shown in FIG. 41.

FIG. 43 is an isometric view of the embodiment of the stirring device shown in FIG. 41, with the cover removed for clarity.

FIG. 44 is an enlargement of a portion of the view of the stirring device shown in FIG. 41.

### DETAILED DESCRIPTION

Embodiments of systems, components and methods of assembly and manufacture will now be described with reference to the accompanying figures, wherein like numerals refer to like or similar elements throughout. Although several embodiments, examples, and illustrations are disclosed below, it will be understood by those of ordinary skill in the art that the inventions described herein extend beyond the specifically disclosed embodiments, examples, and illustrations and can include other uses of the inventions and obvious modifications and equivalents thereof, and combinations of any of the embodiments, features, and details of any of the embodiments disclosed herein with other of the embodiments disclosed herein. Additionally, it should be noted that the descriptions of all of the embodiments disclosed herein should be interpreted to include any of the features, components, and other details of any of the other embodiments disclosed here in combination with or in the alternative to any of the features, components, and other details explicitly described herein.

Additionally, the terminology used herein is not intended to be interpreted in any limited or restrictive manner simply because it is being used in conjunction with a detailed description of certain specific embodiments of the inventions. Also, embodiments of the inventions can comprise several novel features and no single feature is solely responsible for its desirable attributes or is essential to practicing the inventions herein described.

Certain terms may be used in the following description for the purpose of reference only, and thus are not intended to be limiting. For example, terms such as “above” and “below” may refer to directions in the drawings to which reference is made. Terms such as “front,” “back,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

Disclosed herein are embodiments of a processing device (also referred to herein as a processing system, test plate processing system, Petri film card processing system, and the like), that can automate the processing of test cards. Test cards can include, without limitation, Petri film cards, such as but not limited to PETRIFILM™ Plates produced by 3M™. Embodiments of the processing device can be used in a laboratory, office, research facility, testing facility, or any other suitable location. In some embodiments of the processing device disclosed herein, the processing device can be configured to support or secure a petri film card, such as, but not limited to, a 3M PETRIFILM Plate, on a working surface. All suitable culture processing cards, plates, dishes, cell-culture cards, plates, or dishes, Petri dishes, or Petri film cards, including without limitation, the 3M PETRIFILM Plate, or any other similar or suitable culture processing instruments or elements are collectively referred to herein as a sample test plate, or just test plate. In other words, any use of the term test plate in this disclosure can be meant to refer to any suitable culture processing card, plate, dish, cell-culture card, plate, or dish, Petri dish, or Petri film card, including without limitation, the 3M PETRIFILM Plate, or any other similar or suitable culture processing or test processing instrument or element.

For example, any of the embodiments shown or described herein can be configured to work with standard, round Petri dishes. Any of the components disclosed herein can be resized, reshaped, or otherwise reconfigured to work with round Petri dishes or otherwise. Additionally, a plurality of any of the processing device embodiments disclosed herein can be used in tandem and in series to increase the throughput of the system, or in an array on the workspace. For example, a plurality of processor devices can be “daisy-chained” so that an operator could place multiple units in line allowing for multiple test plates to be processed simultaneously, with all of the processors communicating to the system software. The system can be configured to simultaneously process multiple test plates (for example, three test plates, eight test plates, or more). As will be discussed, the system can have a multi-frame or multi-plate holder that is sized and configured to support three test plates or films on a single frame or support member. The system could be configured so that the liquid handlers dispense the test substance into the multiple number of test plates (e.g., 3 test plates, 8 test plates, or more) simultaneously in such a multi-processor arrangement. There is no limit to the number of processors that could be linked together. Such a multiprocessor could have uses in production quality control where many consumables may be tested for manufacturing consistency.

As a brief and nonlimiting overview, any embodiments of the processing device disclosed herein can be used to automate the processing of the use of a test plate for any suitable laboratory test that would use such a test plate. For example and without limitation, any embodiments of the processing device disclosed herein can automate the steps of holding or securing one or a plurality of test plates, whether using test plate frame or otherwise, removing the protective cover of the test plate or otherwise revealing the plating surface, dispensing a predetermined amount of the test solution or sample on the plating surface, and replacing the protective cover of each test plate over the plating surface. Additionally, any embodiments of the processing device disclosed herein can be used to move and position the one or plurality of test plates on a housing member of the processing device, remove each test plate from the housing member of the processing device, and/or move the test plate into an incubator, with any of the aforementioned steps being performed in any desired sequence.

Additionally, as will be described in more detail below, any of the systems disclosed herein can further store a plurality of processed and unprocessed test plates in a storage system, remove each or a plurality of test plates from the storage system, return each or a plurality of test plates to the storage system, and/or even be configured to put the storage plates in an incubator or other further processing device after the storage plates have been processed by the test plate processing device. As used herein, the term processed test plate or processed plate refers to a plate that has received the sample substance on the test plate surface (from a manual pipettor, and automated pipettor, or otherwise) and had the cover or film of the test plate returned back to its original position wherein the film or cover covers the test substance.

PETRIFILM Plates are typically rather small, being smaller than a 3 in×5 inch card, and can be very thin. The relative small size and thin, flexible nature of the PETRIFILM Plates makes the direct handling of such plates by robotic or automation equipment very challenging, if not infeasible and/or impractical. For example, such a consumable can be very difficult for lab robotic system to grasp and/or move around, even if one is using a very expensive fully articulating arm robot, which is not entirely common or cost-effective. In any embodiments disclosed herein, test plate holders can be used to provide an interface with typical robotic arm and hand assemblies by which the test plates can be grasped and moved using typical robotic arm and hand assemblies. However, such holders are not required since other grabbers, devices, or components can be used to automatically move the test plates from one location on the deck to another. To be clear, the holders disclosed herein can be used with any embodiments disclosed herein, but are not required to be used with any embodiments disclosed herein. Additionally, the holder embodiments disclosed herein can be designed to be in the approximate or exact shape of a standard microplate that is routinely used in a testing laboratory and/or otherwise comply with the size and shape requirements of the SBS Standards so that any robotic gripper can grasp the holders. Additionally, the holders can be sized and configured to fit within the registration pins in a typical SBS complying test plate processing system's deck. In any embodiments disclosed herein, the deck can be or can comprise one or more features of the automation liquid handlers deck manufactured by TECAN™.

For example, in any embodiments disclosed herein, a plurality of test plates can each be secured within each of a plurality of test plate holder (also referred to herein as a support frame, test plate frame, plate frame, test plate support member, plate holder, or holding element). The insertion and/or removal of the test plate within the holder can be done manually, or can be automated. The holders can be sized and configured such that the hand or arm (i.e., the gripping member) of a standard automation device can be used to grip the holder so as to move or manipulate the test plate that is coupled with the holder. In other words, the holder can be designed to enable a typical robotic arm to engage with the test plate, whereas the test plate alone (without the holder) would be very difficult if not infeasible for a typical robotic arm to grasp, engage, or couple with. Additionally, a single multi-plate holder can be used to support a plurality of test plates. For example, the multi-plate holder can be configured to support three test plates on one holder, or five or more test plates on one holder.

The robotic arm can then be used to position the holder (collectively referred to herein as either the single plate holder, or the multi-plate holder) on the housing member of the test plate processing device. The overall system can also be configured to insert one or a plurality of the holders supporting the test plate into and/or remove the one or more of the holders from within a storage container, cabinet, or otherwise. Once the holder is positioned on the housing member of the processing device, the processing device can secure the test plate to the housing member of the processing device. For example, without limitation, in any embodiments disclosed suction or a source of low pressure or vacuum can be applied to a portion of the test plate to secure the test plate to the housing member. In any embodiments, suction can be applied through one or more orifices formed through a work surface (also referred to herein as a support surface or stage) of the housing member so that the suction is applied to or in communication with a bottom surface of the test plate to hold the test plate in the desired position on the processing device.

In any embodiments, the housing member of the processing device can be formed from aluminum, steel, or any other suitable material, such as any suitable metal, polymer, and/or composite material, and can have one or a plurality of holes formed through the work surface, the holes being in communication with a suction source so as to provide a vacuum to any objects positioned on the work surface adjacent to or above the holes. Applying the vacuum to the holes can therefore secure the test plate to the work surface while the vacuum is being applied. When it is desired that the test plate be removed from the work surface, the vacuum can be stopped, reduced, or eliminated, thereby removing the suction force that couples the test plate with the work surface.

After the test plate has been coupled with the work surface of the housing member, the processing device can be configured to lift or move the lid or cover of the test plate away from the plating surface of the test plate using a lifting mechanism or element (which can also be referred to herein as a cover moving mechanism or element) so as to expose the plating surface of the test plate. The cover (also referred to herein as a lid) of the test plate can be removed by the processing device using any suitable method, or using any suitable device. For example and without limitation, the processing device can apply suction against the cover or film of the test plate to engage, grasp, or couple with the cover of the test plate, and then can be configured to lift or move the cover away from the plating surface of the test plate. In any embodiments disclosed herein, the processing device can have a suction element or member (which can have one or more suction cups) that can be advanced against the cover of the test plate. Once the suction member or suction element has been advanced so as to be adjacent to or in contact with the cover of the test plate, suction can be applied through the suction member against the cover member so as to couple the suction element with the cover of the test plate. Then, the suction element can then be moved away from the test plate so as to move the cover (or, at least, a portion of the cover) away from the plating surface of the test plate.

In any embodiments disclosed herein, the suction member can be supported by a support member that can be used to move the suction member between a first and a second position, toward or away from the plating surface of the test plate, or toward or away from the cover of the test plate. As used herein, the first position refers to the position of the suction member when a suction member has been moved away from the plating surface of the test plate. The second position is defined as the position of the suction member when the suction member has been moved adjacent to, or in contact with the cover of the test plate, i.e., when the cover of the test plate is in contact with or is adjacent to the plating surface of the test plate.

For example, as will be described in greater detail below, a first portion of the support member can be coupled with a shaft such that the support member can rotate about an axis or centerline of the shaft. The suction member can be coupled with a second portion of the support member. In any embodiments, the second portion of the support member can be a distal portion of the support member such that the suction member can move about an arcuate path relative to the test plate when the support member is moved between a first and a second position. However, the processing device need not be so configured. In any embodiments disclosed herein, the suction member can move along any suitable or desired trajectory or pathway between the first and second positions, including a linear trajectory, a vertically oriented linear trajectory (the vertical direction being defined herein as being normal to the plating surface of the test plate, i.e., parallel with a normal vector), an angled trajectory that is at a positive angle such as, but not limited to, 45 degrees, relative to the normal vector, a horizontally oriented trajectory that is perpendicular to the normal vector, or otherwise.

After the cover of the test plate has been lifted or moved away from plating surface of the test plate, the sample can be pipetted, added to, or otherwise dispensed onto the plating surface of the test plate. The dispensing step can be done manually or, preferably, can be automated. Thereafter, the cover of the test plate can be returned to its covered position by moving the suction member back to the second position, wherein is adjacent to the plating surface of the test plate. The test plate processing system can then move the processed test plate to storage, to another processing device (such as an incubator or spreader), or otherwise. Again, this can include, but is not required to include, spreading the sample solution or material about the plating surface of the test plate using an automated spreader device, moving the test plate to an incubator, moving the test plate to a storage position or container, or performing any other desired steps or further processing of the sample.

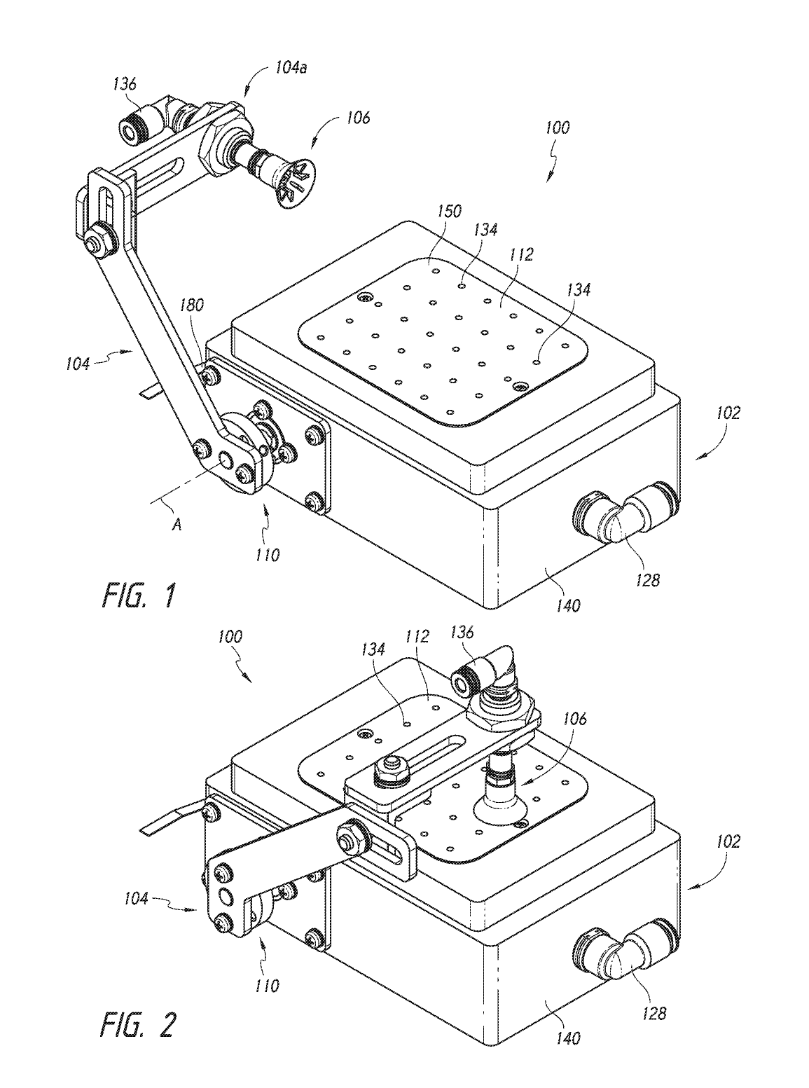

Additional details of some embodiments of the processing device will now be described. FIG. 1 is an isometric view of an embodiment of a processing device 100, showing the support element 104 (also referred to as support member) in a first or open state or position. In any embodiments disclosed herein, the processing device can have a housing member 102, a support element 104 configured to support a suction element 106, and an actuator or actuation element 110. FIG. 1 shows the support element 104 in a first, or open state or position. As illustrated, in the first or open state or position of the processing device, a distal end portion 104a of the support element 104, which can but is not required to have a suction element 106 coupled therewith, is positioned in a first position such that the distal end portion 104a of the support element 104 has been moved away from a working surface 112 of the housing member 102 or processing device 100. In embodiments where suction is used to grasp the film or cover of the test plate, the suction element (such as suction element 106) can be supported by the distal end portion 104a of the support element 104 such that, in the first position or state, the suction element can also be in a position that is spaced apart from the working surface 112 of the processing device 100.

In any embodiments, a distance from the working surface 112 of the processing device 100 to an end portion of the suction element 106 can be approximately 0.75 inch, or from approximately 0.5 inch to approximately 1 inch or more. In any embodiments, a distance from the working surface 112 of the processing device 100 to an end portion of the suction element 106 can be at least approximately 20 times greater than a thickness of a test plate that is positionable on the processing device.

FIG. 2 is an isometric view of the embodiment of the processing device 100 shown in FIG. 1, showing the processing device 100 in a second or closed state or position. As illustrated, in the second or closed state or position, the distal end portion 104a of the support element 104 (which can, but is not required to, have a suction element 106 coupled with the distal end portion 104a) is also in a second state or position wherein the distal end portion 104a and/or the suction element 106 is adjacent to or in contact with the working surface 112 of the housing member 102 or the cover of the test plate supported by the processing device 100.

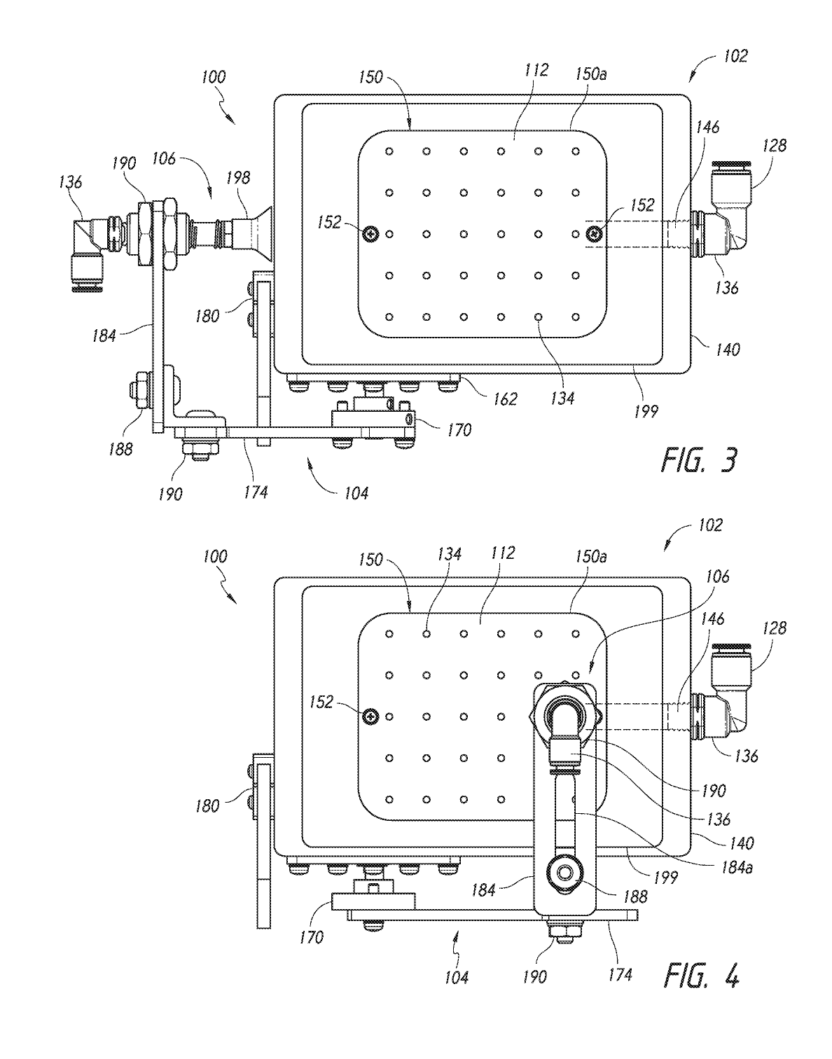

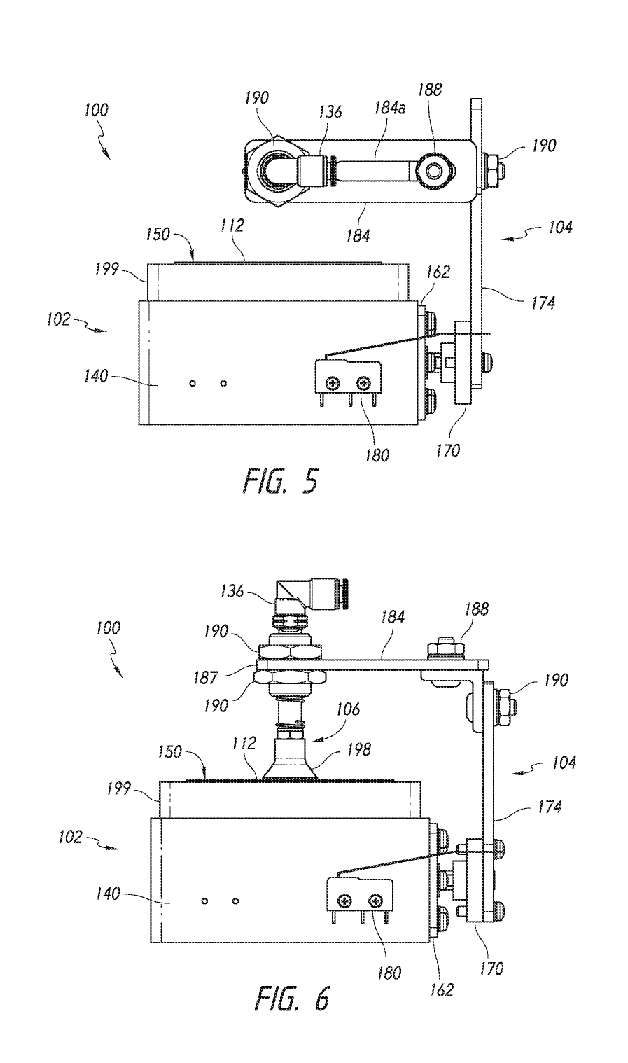

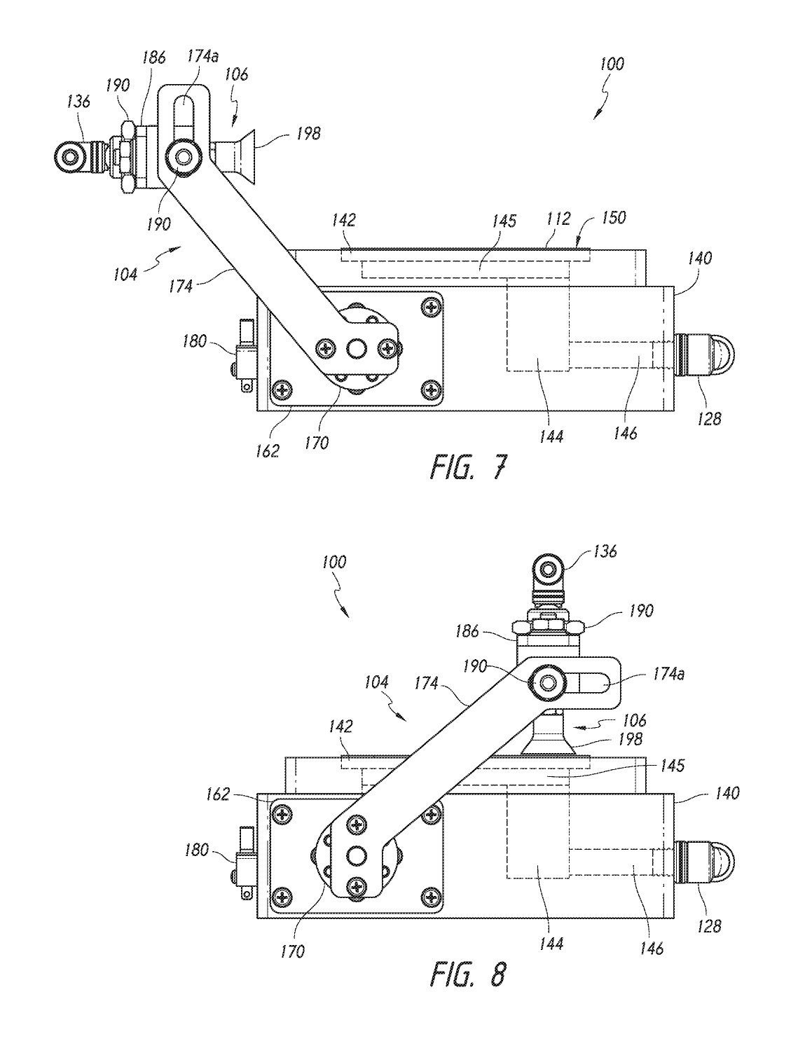

FIGS. 3 and 4 are top views of the embodiment of the processing device 100, showing the processing device 100 and the support element 104 in the first position and the second position, respectively, relative to the housing member 102. FIGS. 5 and 6 are back views of the embodiment of the processing device 100, showing the processing device 100 and the support element 104 in the first position and the second position, respectively, relative to the housing member 102. FIGS. 7 and 8 are side views of the embodiment of the processing device 100, showing the processing device 100 and the support element 104 in the first position and the second position, respectively, relative to the housing member 102.

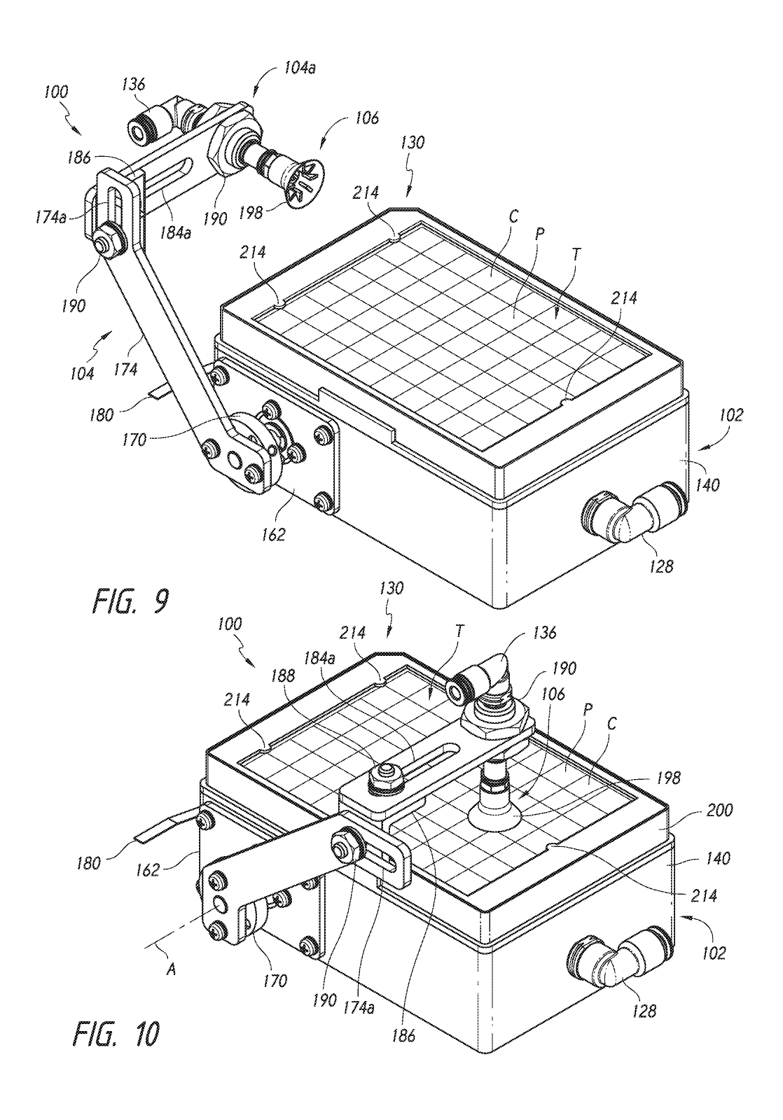

FIGS. 9 and 10 show the embodiment of the processing device 100, showing the processing device 100 and the support element 104 in the first position and the second position, respectively, relative to the housing member 102. In FIG. 10, the suction element 106 is in contact with the cover C of the test plate T while the cover C is adjacent to or in contact with the plating surface P of the test plate T.

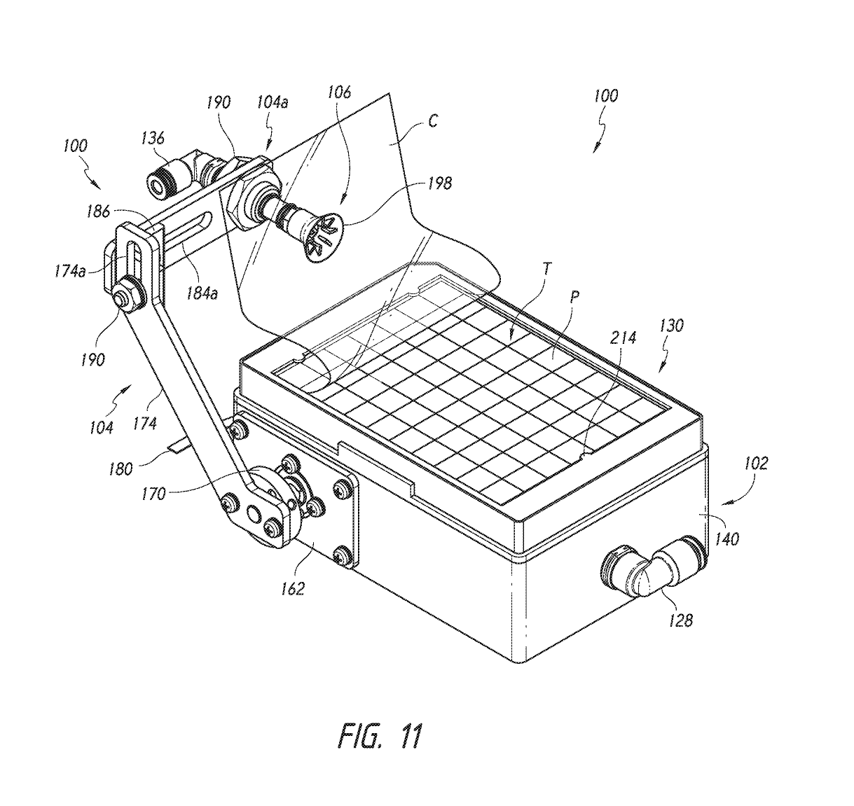

FIG. 11 shows the embodiment of the processing device 100, showing an embodiment of a holder 130 (which has been loaded with a test plate T) positioned on the housing member 102. The test plate support member is also referred to herein as a holder or test plate holder. The suction element 106 is in contact with or is coupled with the cover C of the test plate T, and with the support element 104 and suction element 106 in the first state or position such that the cover C of the test plate T is moved away from the plating surface P of the test plate T. In any processing device or system embodiments disclosed herein, the suction element 106 can be replaced with any other suitable capture element to releasably connect with the test plate cover or film. The capture element can have a static attraction element, a sticky element or substance, or other element or component that can grasp, support, or couple with the cover or cover film of the test plate in addition to or in place of the suction element of any embodiment disclosed herein such that any description of the suction element disclosed herein can be substituted with any suitable capture element. Note that, even though FIGS. 9-11 illustrate holder embodiment 130, holder embodiment 230 shown in FIGS. 17-18 can be used in place of holder embodiment 130 in any of the processing device and processing system embodiments disclosed herein.

One embodiment of a method of using the processing device will now be described. In any embodiments disclosed herein, additional steps can also be performed, and/or some of the steps described below can be omitted. Additionally, one or more of these steps can be performed in any desired sequence that is different than what is described below. Therefore, even though the steps are described in a particular order or even designated to be within a particular order, the sequence of the steps listed below is not required, and the steps can be performed in any other desired order. Additionally, in any method or apparatus embodiments disclosed herein, one or more of the steps or sequences can be performed simultaneously.

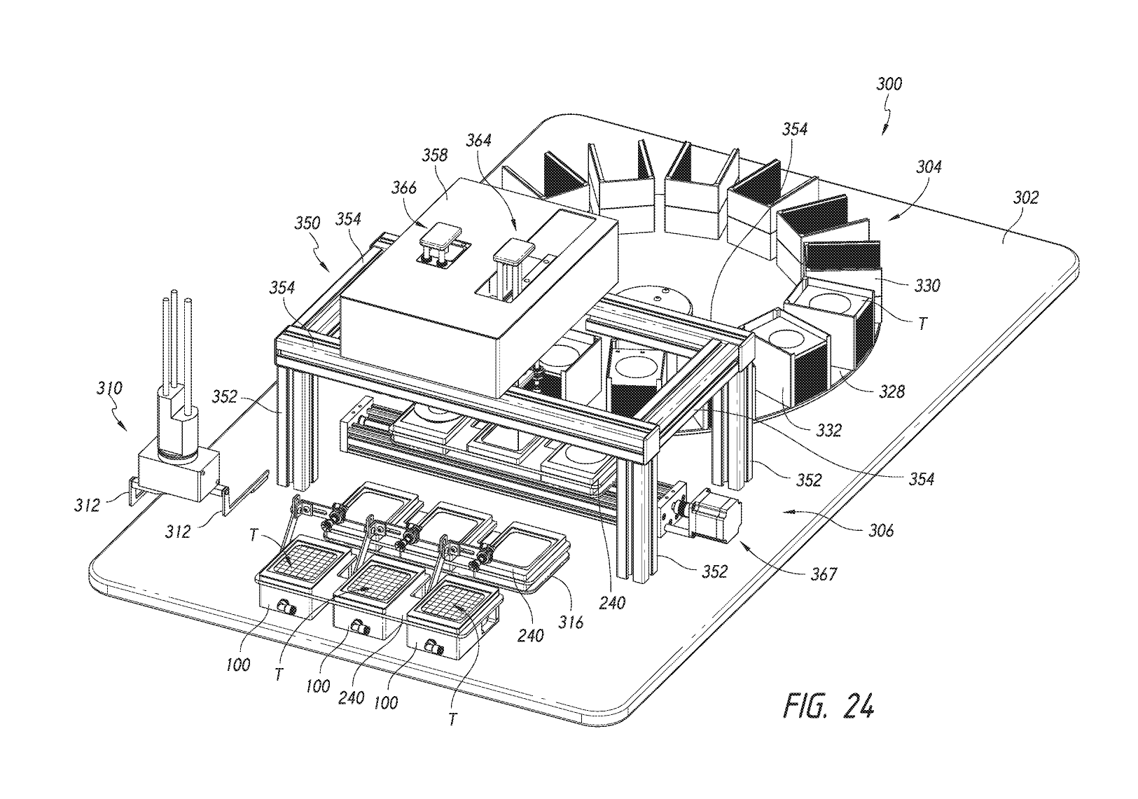

In use, the support element 104 can be positioned in, or moved to, the first position, as shown in FIG. 1. This permits the generally greater access to the working surface 112 of the housing member 102 such that the holder 130 supporting a test plate T (such as, but not limited to, test plate T shown in FIGS. 9-11) can be positioned adjacent to the working surface 112 of the housing member 102, as shown in FIG. 9. In any embodiments disclosed herein, the holder 130 can be positioned on the housing member 102 in any manual or automated fashion. For example and without limitation, a robotic arm or gripper mechanism such as the robotic gripper or arm 310 shown in FIG. 24, can be used to grasp or support the holder 130 supporting the test plate T, to position the holder 130 adjacent to the working surface 112 of the housing member 102, and then to release the holder 130 when the holder 130 is in the desired position relative to the working surface 112 of the processing device 100. Additionally, as is shown in FIG. 24, the robotic arm 310 can be used to load a multi-plate holder 330 filled with a plurality of test plates T onto a plurality of processing devices 100 by either grasping the multi-plate holder 330 or lifting the multi-plate holder 330 from the bottom side of the multi-plate holder 330.

Suction or reduced pressure can be applied through the coupling or connector 128 from a pump, reduced pressure reservoir, fan driven suction device, or other source of reduced pressure to the openings 134 projecting through the working surface 112 of the housing member 102 and against the bottom surface of the test plate T at a sufficient pressure level to secure the test plate T in the desired position relative to the working surface 112 of the housing member 102. The suction or reduced pressure can be applied to the test plate T either before or after the robotic arm has released the holder (which can be holder 130 or multi-plate holder 330).

As shown in FIG. 9, the cover C of the test plate T can remain in the first or initial position as the robotic arm or other mechanism releases the holder 130. Thereafter, the processing device 100 can be moved to the second state or position, which can involve moving the support element 104 so as to move the suction element 106 in contact with the cover of the test plate when the cover C of the test plate T is in the first position (i.e., when the cover C of the test plate T is positioned adjacent to or in contact with the plating surface P of the test plate T). At that point, suction can be applied via the suction element 106 to the cover C of the test plate T (or other gripping mechanism can be actuated) so as to couple the cover C of the test plate T with the distal portion 104a of the support member 104 so that, when the distal end portion 104a of the support member is moved away from the working surface 112 of the processing device, the cover C can be lifted away from the plating surface P of the test plate T. Suction or reduced pressure can be applied through the suction element 106 against the cover C of the test plate T using a pump, reduced pressure reservoir, fan driven reduce pressure device, or other suitable device in communication with the coupling or connector 136 of the suction element 106.

As mentioned, after suction has been applied to the suction element 106 so as to couple the cover C of the test plate T with the suction element 106, the support element 104 and, consequently, the suction element 106, can be moved from the second position toward the first position so as to lift the cover C away from the plating surface P of the test plate T as shown in FIG. 11. With the cover C moved away from the plating surface P of the test plate T (i.e., with the processing device 100 moved to the first position), the plating surface P is now accessible for dispensing the desired substance onto the plating surface P. The desired substance can be dispensed onto the plating surface P using an automated pipetting device or system, or the substance can be dispensed to the plating surface P manually.

In any embodiments disclosed herein, an automated pipetting component or device can be used to deliver the desired amount, arrangement, and positioning of the test substance to the plating surface P of the test plate T. For example and without limitation, the automated pipetting device can deliver an array of droplets of the desired substance onto the plating surface P of the test plate T in a circular, gridlike, or other arrangement that, when the cover C of the test plate T has been replaced or moved back to the closed position, the test substance on the plating surface P will be suitably arranged such that no additional spreading or spreader step will be required to spread the test substance about the plating surface P. This can improve the efficiency of the overall system by eliminating the spreader step and allowing the test plate T to go directly to an incubator or other subsequent processing device, or to the storage system or storage device.

Thereafter, the processing device 100 can then be moved to the second position by moving the distal end portion 104a of the support element 104 and suction element 106 to the second position so as to return the cover C of the test plate T back to the closed position wherein the cover C is adjacent to and covers the plating surface P of the test plate T. Once the cover C has returned to the first position, the reduced pressure or suction applied to the cover C through the suction element 106 can be reduced or eliminated to decouple or release the cover C from the suction element 106 or distal end portion 104a o the support member 104. The distal end portion 104a can be returned to the first position such that the holder 130, multi-plate holder 330, and/or test plate T can be removed from the processing device 100.

Thereafter, the processing system can perform further processing of the test plate or further steps can be performed manually, or a combination of both. For example, the substance can be (but is not required to be) spread about the plating surface P of the test plate T using a scraper, a squeegee, a roller, a press, or any other suitable device or technique. Once all processing that is desired to be completed while the holder 130 is supported by the housing member 102, the suction or reduced pressure applied through the coupling 128 and the openings 134 can be reduced or eliminated. Thereafter, the holder 130 supporting the test plate T can be moved away from the housing member 102 using a robotic arm or otherwise, or manually, into storage, into an incubator, or into any other desired position. The entire process can then be repeated as many times as is desired.

In a typical laboratory setting, a single processor device can be configured to process approximately 100 samples per day, while it is not uncommon for laboratories to require the processing of 800 or more test plates per day. The following is a non-limiting example of the workflow for a processing device, including any of the embodiments of the processing device disclosed herein. Any of these steps can be omitted from this workflow and/or additional steps can be introduced into this workflow, and one or more of any of these steps can be performed simultaneously. This is meant as an example and not as a requirement of the workflow of any processing device embodiments disclosed herein.
1) retrieve the test plate from storage, which can have a storage hotel (also referred to just as a hotel);
2) position the test plate on the processing device (for example, by using a robotic gripper or gripper and holder, as described herein);
3) engage the film of the test plate, which can comprise moving a distal end portion of the support member of the processing device having a grasping mechanism toward the test plate;
4) move the film or cover of the test plate away from the working surface of the test plate, which can comprise moving the distal end portion of the support member away from the test plate;
5) deliver a subject sample to the working surface of the test plate using a pipettor or other suitable instrument;
6) move the film back into contact with the working surface of the test plate to cover the subject sample, which can comprise moving the distal end portion of the arm back toward the working surface of the test plate;
7) spread the subject sample about working surface of test plate (for example, a robotic gripper can pick up a spreader, deliver the spreader to the processing device, and press down on the test plate to spread the subject sample, and then return the spreader to its storage position, or the test plate can be moved to be in alignment with a spreader device);
8) move the test plate using, for example, a robotic gripper, holder, or otherwise to a subsequent processing device, back to storage, or otherwise.

In some embodiments of the processor device, this entire workflow for one test plate can be completed start to finish in approximately 60 seconds. Some embodiments can be configured to complete the entire workflow in approximately 50 seconds to approximately 70 seconds, or from less than approximately 45 seconds to more than approximately 75 seconds. Using this workflow, any embodiments of the processor device can process approximately 60 test plates per hour (approximately 480 test plates per 8 hour period). Some embodiments can be configured to process from approximately 50 to approximately 70 test plates per hour (from approximately 400 to approximately 560 test plates per 8 hour time period).

Additionally, using the multi-plate holder 330, multiple processing devices, and the other components used to load and unload the test plates from the holders and to move the holders around the processing deck, the processing time per test plate can be significantly reduced. For example and without limitation, using the multi-plate holder 330, multiple processing devices, storage hotels, and other robotic devices to process multiple test plates simultaneously, the processing time per test plate can be as low as approximately 15 seconds or less, or from approximately 15 seconds to approximately 25 seconds per test plate, or from approximately 25 seconds to approximately 30 seconds per test plate.

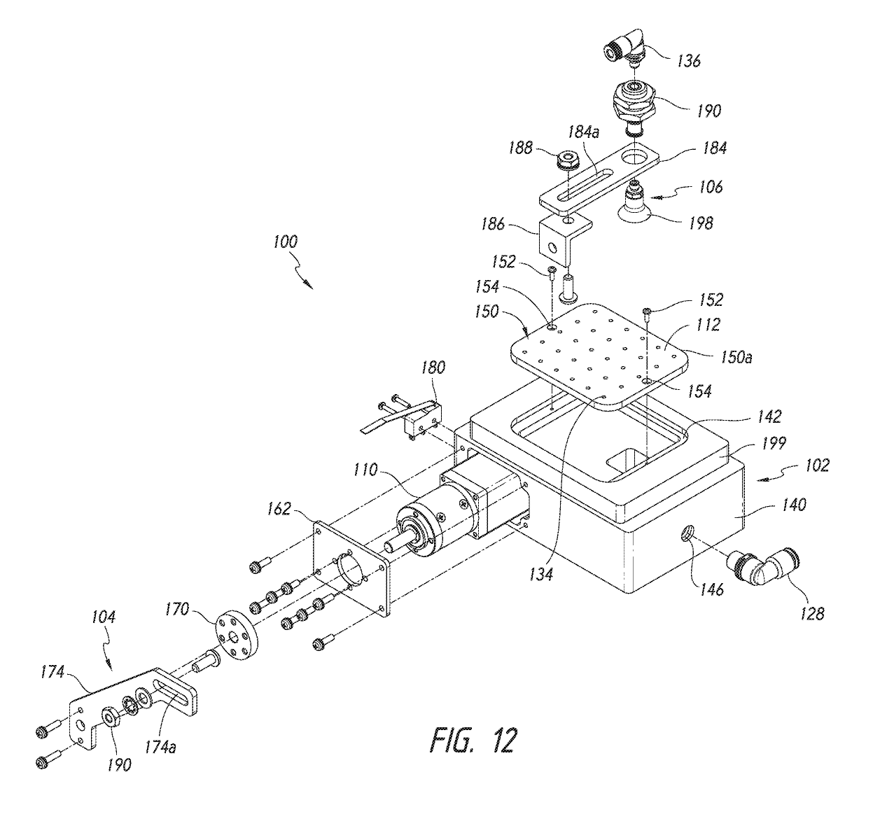

More details regarding the embodiment of the processing device 100 will be described with reference to FIG. 12, which is an exploded view of the processing device 100. The housing member 102 can have a body member 140 that can be made from any suitable material, including aluminum, steel, or any other suitable metallic, polymeric, or composite material, and/or any other combination thereof. The body member 140 can have a recess 142 formed in or extending through a top surface 140a of the body member 140. The recess can be sized and configured such that the base plate 150 (also referred to herein as a base member) can be coupled with and supported within the recess 142. The space between the recess 142 and the base plate 150 can be sealed so as to reduce or eliminate any air or fluid passageways around a perimeter or outside edge 150a of the base plate 150. Sealing the perimeter or outside edge 150a of the base plate 150 can increase the efficiency of the suction or reduced pressure applied through the openings 134 in the base plate 150. Additionally, though not required, the body member 140 can have an outside or perimeter ledge or recess 199 sized to receive the holder or portion of the multi-plate holder so that the holder or multi-plate holder is secured in the horizontal directions also. Please note that any of the components disclosed herein can be sized and configured to satisfy or comply with SBS Standards so as to be easily integratable with a standard robotic systems workspace or deck.

With reference to FIG. 14, which is an isometric view of an embodiment of the body member 140, the body member 140 can have an air passageway 144 formed therein, the air passageway being in communication with an opening 146. The opening 146 can be threaded such that the coupling member 128 can be threadedly engaged with the opening 146. All connections can be sealed to reduce leaks or undesired air passageways. A second recess or air passageway 145 can be formed in the body member 140 such that the vacuum or reduced pressure applied through the coupling 128 and through the opening 144 can also be communicated through the opening or recess 145 such that reduced pressure can be applied to all or substantially all of the openings 134 formed in the base plate 150.

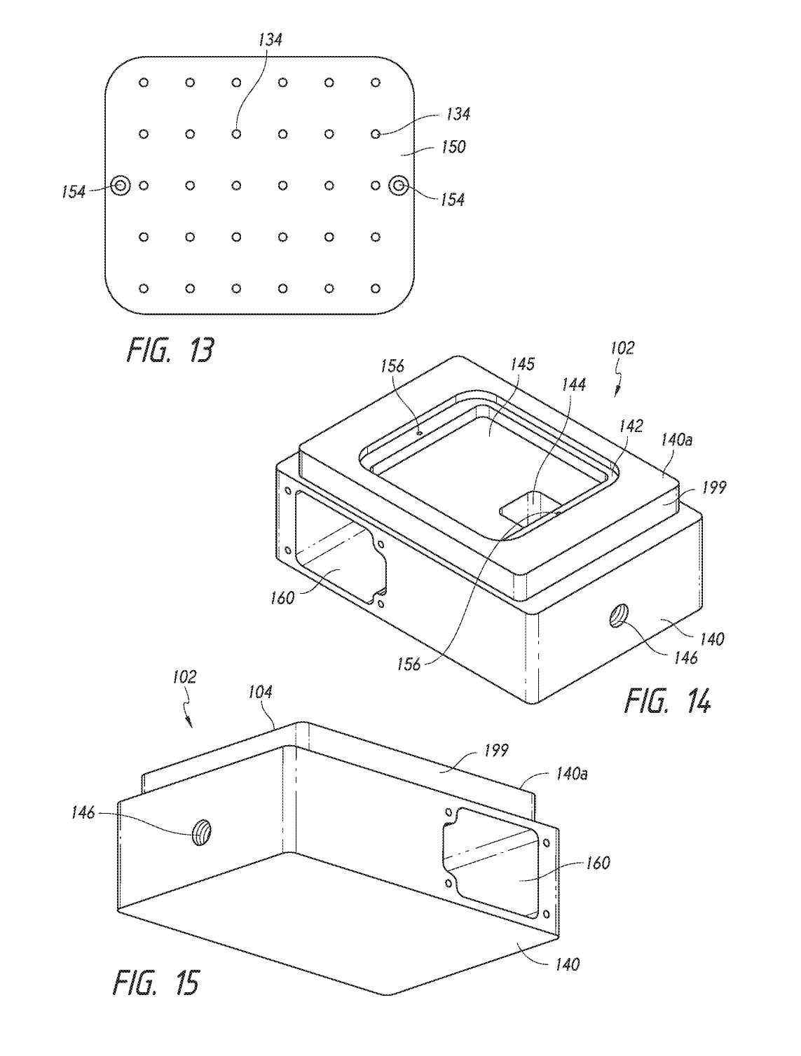

In any embodiments disclosed herein, the base plate 150 can have any suitable size and shape, including the size and shape illustrated in FIG. 13. Additionally, the base plate 150 can have any desired number of openings 134 formed therein to ensure adequate reduced pressure or suction is applied to the bottom surface of the test plate T. For example and without limitation, the base plate 150 can have thirty holes as shown in FIG. 13. Alternatively, in any embodiments, the base plate 150 can have between four and fifty or more openings or between ten and forty openings, or any desired number of openings in any desired or suitable arrangement. Fasteners 152 can be advanced through openings 154 formed in the base plate 150 so as to threadably engaged with the threaded openings 156 formed in the body member 140 to secure the base plate 150 to the body member 140.

Additionally, as shown in FIG. 14, an opening 160 can be formed in the body member 140 to accommodate or house an actuator, such as actuator 110. A cover plate 162 can be used to secure the actuator 110 to, or couple the actuator 110 with the body member 140. As shown, fasteners can be advanced through openings in the cover plate 162 into the actuator and also into the body member 140 to secure the actuator 110 to the body member 140. In any embodiments disclosed herein, the actuator 110 can be a gear motor, such as the Anaheim Automation 11YPG Series High Torque Stepper Gear Motor or any motor that is similar to or equivalent to the Anaheim Automation gearmotor. In any embodiments disclosed herein, the actuator 110 can be a direct drive servo motor, such as the P Series Servo Motor by the Parker Hannifin Corporation. In any embodiments disclosed herein, the actuator 110 can be a stepper motor.

A mounting hub 170 can be fixed to the shaft of the actuator to couple the support element 104 with the actuator 110. In particular, fasteners can be advanced through openings formed in a first support member 174 of the support element 104 and threadably engaged with the mounting hub 170 so as to couple the first support member 174 with the mounting hub 170 and, consequently, the shaft of the actuator 110. In the coupled configuration, a rotation of the shaft of the actuator 110 will cause the simultaneous and consequential rotation of the first support member 174. The mounting hub 170 can be custom-made, or can be purchased off-the-shelf. For example, an aluminum mounting hub for a 6 mm shaft sold by Pololu Robotics and Electronics can be used.

Any embodiments of the test plate processing device can have a limit switch 180 configured to limit a rotation of the support element 104. In some embodiments, the limit switch 180 can be mounted on or otherwise coupled with the body member 140. In some embodiments, the limit switch 180 can be mounted on or otherwise coupled with the support member 104, or on any other component of the processing device or otherwise. For example, as shown most clearly in FIG. 1, the switch 180 can be positioned such that, when the first support member 174 reaches the desired angular orientation (in the first position), the limit switch will be triggered so as to signal to the actuator to stop the rotation of the support element 104. For example and without limitation, a Snap-Action Switch with 50 mm Lever supplied by Pololu Robotics and Electronics can be used. In any embodiments disclosed herein, the support element can rotate about an angle that is approximately 90 degrees, or from approximately 30 degrees to approximately 110 degrees, or from approximately 60 degrees to approximately 90 degrees. The angle can be increased if additional clearance is needed in the first position between the processing device components and the test plate. The angle can be decreased to increase efficiency of the system.

Any processing device 100 embodiments can alternatively or additionally use an encoder or other control system device or component with the actuator to control a position of the support member 104. In any embodiments, the second position of the support member can be controlled by a motor controller and/or software control.

A second support element 184 can be coupled with the first support member 174. In any embodiments disclosed herein, a bracket member 186 can be used to couple the first and second support elements 104, 184. Additionally, in any embodiments disclosed herein, the first and second support elements can be formed monolithically. However, by forming the support element 104 with the first and second support elements 174, 184, the operator can adjust the relative position of the first and second support elements so as to adjust the position and/or angular orientation of the suction element 106 to achieve the optimal positioning of the suction element 106 for the operation of the processing device 100. For example, positioning slots 174a and 184a can be formed and positioned in the first and second support elements 174, 184 to permit the adjustability of the relative position of the first and second support elements 174, 184. The threaded fastener 188 and a threaded fastener 190 can be used to couple the first and second support elements 174, 184, together.

Additionally, with reference to FIG. 12, a leveler 190 (which can be a spring leveler, such as the VSL1-20 Spring Leveler supplied by Vaccon) or other similar or suitable leveling element or component can be used to couple the suction element 106 to the support element 104. The leveler 190 can be used to orient the suction element 106 so that the suction element 106 is approximately flat against or perpendicular relative to the cover C of the test plate T. In any embodiments, the suction element can comprise one or more suction cups. Any suitable suction cup or vacuum cup can be used, including, without limitation, the Flat Vacuum Cup VCR-F15P supplied by Vaccon.

In any embodiments disclosed herein, the support element 104 and, consequently, the suction element 106 or other grasping element, which can be used in place of any of the suction elements in any embodiments disclosed herein, can move along any suitable or desired trajectory or pathway between the first and second positions, including a linear trajectory, a vertically oriented linear trajectory (the vertical direction being defined herein as being normal to the plating surface of the test plate, i.e., parallel with a normal vector), an angled trajectory that is at a positive angle relative to the vector, a horizontally oriented trajectory that is perpendicular to the normal vector, or otherwise. For example and without limitation, in the embodiments described herein, the support element 104 can be configured to rotate about an axis A (shown in FIG. 1) that projects through the centerline of the shaft connected to an actuator 110. The actuator can be a gearmotor, for example.

Additionally, electrical and/or pneumatic circuits can be supported by the housing member 102. Pressure regulators or a dual pressure regulator can be used to regulate a pressure of the suction applied both to the bottom surface of the test plate, but also to regulate a pressure of the suction applied to the suction member that is applied to the cover C of the test plate. In any embodiments, the operating pressure of the suction element 106 can be approximately 60 psi, or from approximately 50 psi to approximately 70 psi, or to approximately 80 psi. Additionally, pressure can be applied to the base plate to be communicated through the openings 134 at approximately 80 psi.

In any embodiments, the body member 102 can be approximately 5.0 inches long by approximately 3.4 inches wide. The body member 102 can be approximately 1.9 inches tall. Note that these are representative or exemplifying dimensions of the body member 102. The body member 102 is not limited to the sizes or dimensions, and can be any suitable size depending on the size of the test plate being used, and/or the spacing available on the automation deck.

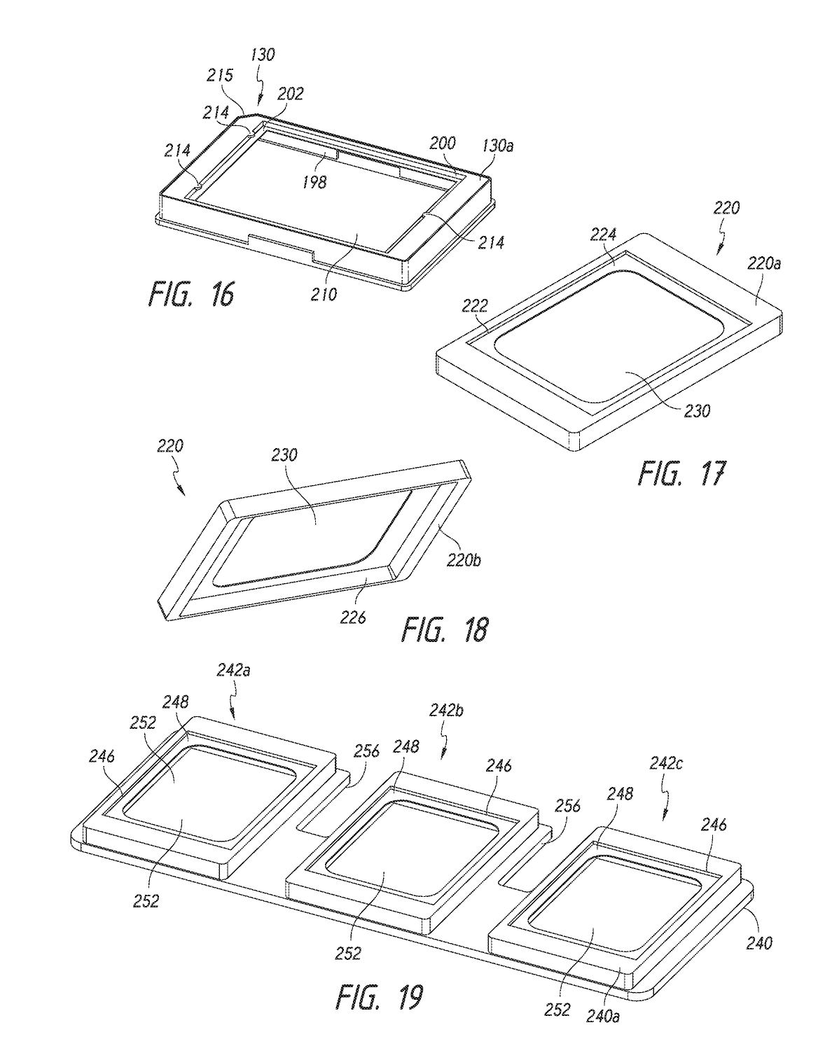

FIG. 16 is an isometric view of the embodiment of the test plate holder 130 of the embodiment of the processing device shown in FIG. 9. As described herein, the embodiment of the test plate holder 130 disclosed herein can be configured to support a 3M PETRIFILM™ Plate so as to enable a robotic arm or other robotic component to be coupled with or grasp the holder 130 so that a test plate T supported by the holder 130 can be manipulated by the automation components.

With reference to FIG. 16, the holder 130 can have a first main recess 198 formed therein, the recess 198 being sized and configured to fit about an around the complementary portion of the body member 140 of the housing member 102. For example, with reference to FIGS. 12 and 14, a ledge or recess 199 can be formed around a perimeter of the body member 140, the recess or ledge 199 being sized and configured to be complementary to the recess 198 formed in the holder 130.

The holder can have a recess 200 formed through a top surface 130a of the holder 130. A ledge or backing surface 202 can be formed so as to be approximately parallel with the top surface 130a of the holder 130. The ledge 202 can provide a surface against which a test plate T can be secured. An opening 210 can be formed through the holder 130. The opening 210 is sized and configured such that the source of reduced pressure or vacuum communicated through the openings 134 in the base plate 150 can be applied to a bottom surface of a test plate T supported within the holder 130 through the opening 210. One or more tabs 214 can be positioned adjacent to the recess 200 to secure the test plate T to the holder, without preventing or obstructing the cover C from being removable or movable away from the plating surface P of the test plate T.

FIG. 17 is an isometric view of another embodiment of a test plate holder 220 that can be used with any of the processing devices 100 disclosed herein, and can have any of the same features, capabilities, and/or other details as the holder 130. FIG. 18 is an isometric view of the holder 220 shown in FIG. 17, showing an underside of the holder 220. One difference between holder 220 and holder 130 is that the tabs 214 that are formed on or positioned on holder 130 are not included on holder 220. As such, the embodiment of the test plate holder 220 disclosed herein can be configured to support a 3M PETRIFILM™ Plate so as to enable a robotic arm or other robotic component to be coupled with or grasp the holder 220 so that a test plate T supported by the holder 220 can be manipulated by the automation components and moved to and from the processing devices 100.

With reference to FIG. 18, the holder 220 can have a recess 222 formed through a top surface 220a of the holder 220. A ledge or backing surface 224 can be formed so as to be approximately parallel with the top surface 220a of the holder 130. The ledge 224 can provide a surface against which a test plate T can be secured. The holder 220 can have another recess 226 (referred to also as a bottom recess or second recess) formed through a bottom surface 220b of the holder 220, the recess 226 being sized and configured to fit about an around the complementary portion of the body member 140 of the housing member 102. In some embodiments, the recess 226 can have tapered walls, with the opening or recess being the largest adjacent to the bottom surface 220b. The tapered walls can make it easier to couple and align the holder 220 with the processing devices 100.

An opening 230 can be formed through the holder 220. The opening 230 is sized and configured such that the source of reduced pressure or vacuum communicated through the openings 134 in the base plate 150 can be applied to a bottom surface of a test plate T supported within the holder 220 through the opening 230. The reduced pressure can hold the test plate T in the desired position during processing of the test plate T.

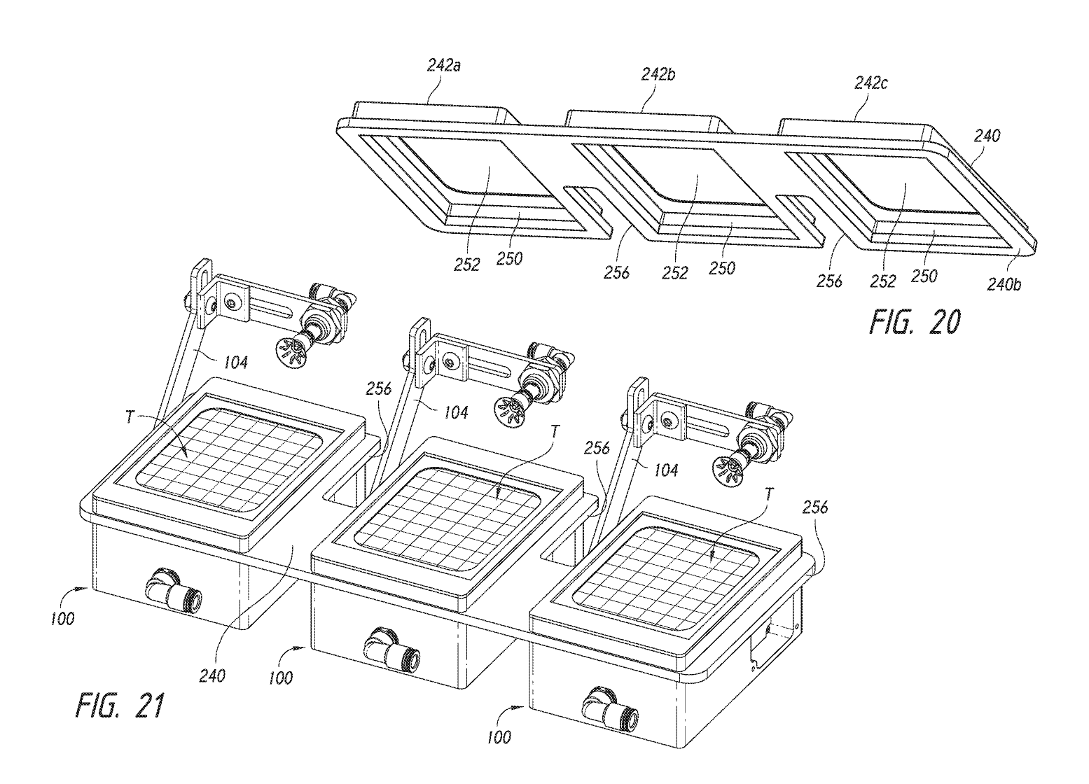

FIGS. 19 and 20 are isometric views of an embodiment of multi-plate holder 240 that can be used with any arrangement of a plurality of the processing devices 100 disclosed herein. FIG. 21 is an isometric view of the embodiment of the multi-plate holder 240 of FIG. 19, positioned on a series of processing devices 100. The embodiment of the multi-plate holder 240 illustrated in FIGS. 19-21 can have a first test plate holder portion 242a, a second test plate holder portion 242b, and a third test plate holder portion 242c. Each portion 242 (i.e., each of 242a, 242b, and 242c) of the multi-plate holder 240 can support a test plate T and can have any of the same features, capabilities, and/or other details as the holder 130 and/or holder 220. Some embodiments of the multi-plate holder 240 can be formed as a single piece. Some embodiments of the multi-plate holder 240 can be formed as separate pieces and coupled together. For example, without limitation, the raised portions of the holder 240 that are similar to the holders 220 can be formed monolithically with the base plate portion of the multi-plate holder 240, or can be formed separately and bonded or otherwise irreversibly coupled together.

The embodiment of the multi-plate holder 240 disclosed herein can be configured to support a plurality of 3M PETRIFILM™ plates so as to enable a robotic arm or other robotic component to be coupled with or grasp the multi-plate holder 240 so that the plurality of test plates T supported by the multi-plate holder 240 can be simultaneously manipulated by the robotic and/or automation components and moved to and from the processing devices 100. Performing at least some of the processing steps of multiple test plates simultaneously can significantly improve the operating efficiency and significantly reduce the processing time of the processing system embodiments disclosed herein. In the illustrated example, the multi-plate holder 240 has three test plate support portions 242 and can simultaneously support between one and three test plates T. However, in any embodiments disclosed herein, the multi-plate holder can be configured to support any desired number of test plates by reducing the number of test plate support portions 242 or by increasing (i.e., duplicating) the number of test plate support portions 242.

With reference to FIG. 19, each test plate holder portion 242 of the multi-plate holder 240 can have a recess 246 formed through a top surface 240a of the multi-plate holder 240. A ledge or backing surface 248 can be formed in each test plate holder portion 242 that has a planar surface that is approximately parallel with the top planar surface 220a of the multi-plate holder 240. The ledge 248 can provide a surface against which a test plate T can be secured. The multi-plate holder 240 can have another recess 250 (referred to also as a bottom recess or second recess) formed through a bottom surface 240b of the multi-plate holder 240 in each portion 242 of the multi-plate holder 240. The recess 250 of each portion 242 of the multi-plate holder 240 can be sized and configured to fit about an around the complementary portion of the body member 140 of the housing member 102.

One or more openings 252 can be formed through the multi-plate holder 240. The openings 252 can be sized and configured such that the source of reduced pressure or vacuum communicated through the openings 134 in the base plate 150 of each processing device can be applied to a bottom surface of a test plate T supported within the multi-plate holder 240 through the openings 252. The reduced pressure can hold the test plate T in the desired position during processing of the test plate T. In some embodiments, the recess 250 in each portion 242 can have tapered walls, with the opening or recess being the largest adjacent to the bottom surface 240b. The tapered walls can make it easier to couple and align the multi-plate holder 240 with the processing devices 100 that have been arranged in a spatial arrangement that matches the positions of each of the recesses 250 of the multi-plate holder 240, as shown in FIG. 21. Additionally, the multi-plate holder 240 can have a cutout 256 formed in the multi-plate holder 240 between each of the portions 242 of the multi-plate holder 240 to permit the support member 104 of each processing device 100 to move freely between the first and second positions without obstruction by the multi-plate holder 240.

Any of the holder and multi-plate holder embodiments disclosed herein can be made from any suitable material, and is preferably rigid to prevent the holder from bending too much when grasped or lifted by a robotic gripper, arm, or other component. Any of the holder or multi-plate holder embodiments can have an angled corner, such as angled corner 215 of the holder 130 show in FIG. 16, for providing an indexing reference for the holder. This angled corner can be sized and configured to be consistent with a standard microplate's dimensions, and/or can ensure that the holder will work with standardized microplate hotels (storage units).

Additionally, in any embodiments disclosed herein, a single processing device having an extended housing and a plurality of working surfaces can be used in place of the plurality of independently operable processing devices. The single, multi-station processing device can be configured to receive and support a multi-plate holder 240 having multiple test plates thereon. For example and without limitation, the housing of the multi-station processing device can have a single continuous housing that has three sets of base plates 150. In some embodiments, each of the three base plates 150 can be supplied with suction through an interconnected air passageway through the continuous housing. Additionally, a single support element (similar to support element 104) can support three separate suction elements 106 at each of the three respective working stations on the processing device such that only one actuation device in total would be needed to process three test plates simultaneously. This would simplify the system and potentially reduce the cost of the system.

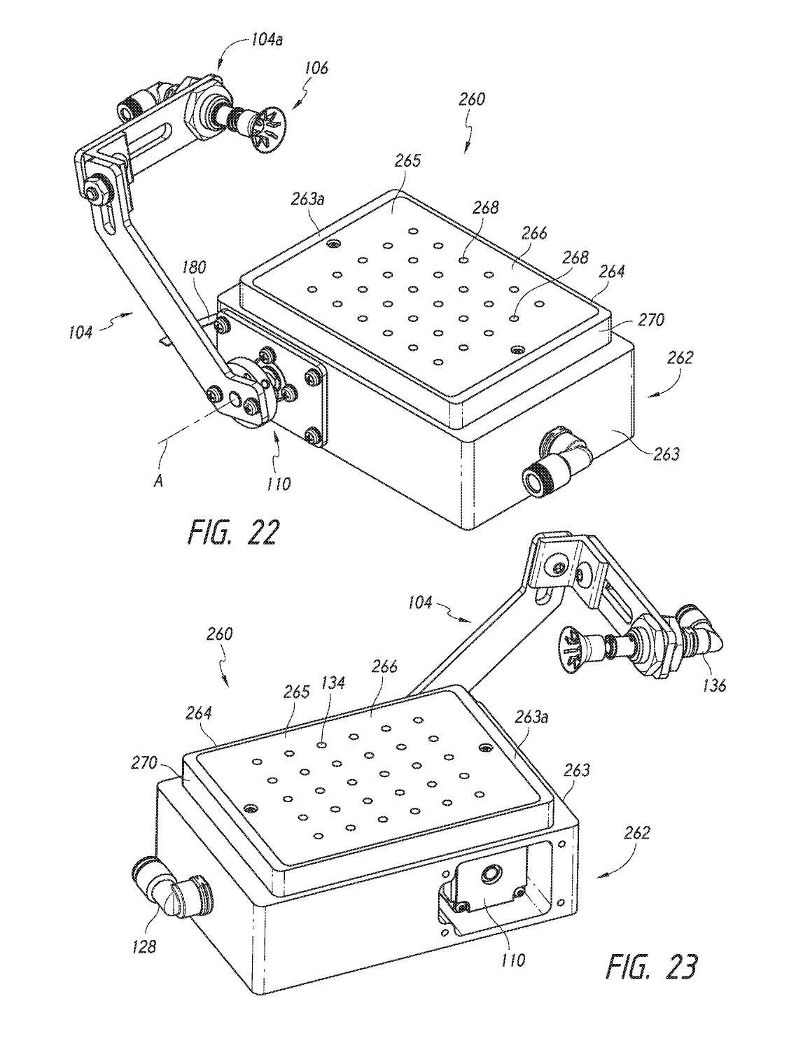

FIGS. 22 and 23 are isometric views of another embodiment of a test plate processing device 260, showing the processing device 260 in a first or open state or position. Any embodiments of the processing device 260 disclosed herein can have any of the same features, components, sizes, materials, or other details of any of the other processing device embodiments disclosed herein, including, without limitation, the processing device 100, in addition to, in the alternative to, and/or in combination with any of the other features, components, sizes, materials, or other details described herein with respect to processing device 260. Additionally, any embodiments of the processing device 100 disclosed herein can have any of the features, components, sizes, materials, or other details of processing device embodiment 260 disclosed herein, in addition to, in the alternative to, and/or in combination with any of the other features described with respect to processing device 100 herein.

The housing member 262 of processing device 260 can have a body member 263 that can be made from any suitable material, including aluminum, steel, or any other suitable metallic, polymeric, or composite material, and/or any other combination thereof. The body member 263 can have a recess 264 formed in or extending through a top surface 263a of the body member 263. The recess 264 can be sized and configured such that the base plate 265 can be advanced into and supported within the recess 264. The space between the recess 264 and the base plate 265 can be sealed so as to reduce or eliminate any air or fluid passageways around a perimeter or outside edge 265a of the base plate 265. Sealing the perimeter or outside edge 265a of the base plate 265 can increase the efficiency of the suction or reduced pressure applied through the openings 268 in the base plate 265. Additionally, though not required, the body member 263 can have an outside or perimeter ledge or recess 270 sized to receive the holder or portion of the multi-plate holder so that the holder or multi-plate holder is secured in the horizontal directions also. Please note that any of the components disclosed herein can be sized and configured to satisfy or comply with SBS Standards so as to be easily integratable with a standard robotic systems workspace or deck.

Any embodiments of the processing device 260 can have a support element 104, a suction element 106 or other capture element to capture or grasp the test plate cover or film, an actuator 110, a limit switch 180, and/or any of the tubing components, coupling components, air passageways, and other features, components and details of any other test processing embodiment disclosed herein.

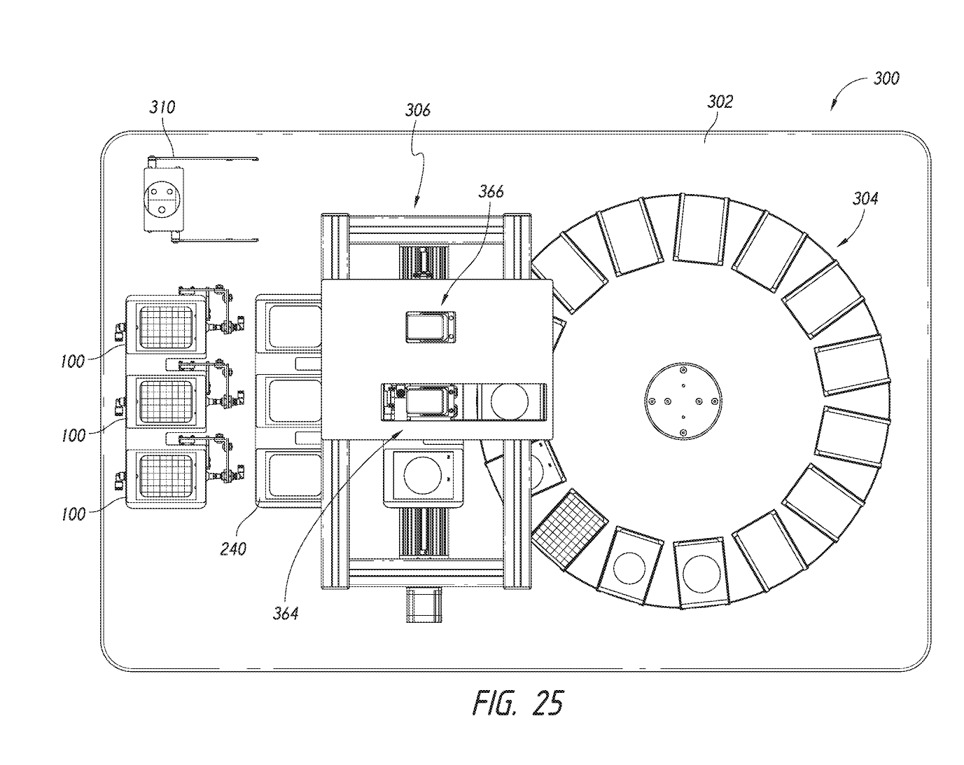

FIGS. 24-25 are an isometric view and a top view of an embodiment of a processing system 300 having a working surface or deck 302, a test plate storage device 304, a test plate handling system 306, and a plurality of test plate processing devices 100. The test plate processing devices 100 of the processing system 300 can have any of the features, components, or details of any of the test processing devices disclosed herein.

Additionally, embodiments of the processing system 300 can have a robotic arm or grip 310 that can be used to move any of the components of the processing system 300 around or adjacent to the deck 302, or beyond the deck if desired. The robotic gripper 310 can be configured to move in any horizontal and vertical directions, and can rotate about at least a vertical axis (i.e., an axis that is normal to the surface of the deck 302). Additionally, the robotic gripper 310 can have grippers or tines 312 that move inward and outward (i.e., toward and away from each other) so as to grasp objects, as desired. One example of a robotic arm or grip that can be used for at least some embodiments of the processing system disclosed herein is the robotic arm manufactured by TECAN™ that is sometimes offered as part of the Freedom Genesis or EVO® platforms.

Some embodiments of the processing system 300 are configured to be fully automated, once the test plates have been loaded into the storage system 300. The processing system 300 is configured so that the processing system can move test plates and/or storage modules to and from the processing devices 100 and/or other post-processing devices, such as incubators, spreaders, and/or storage devices, and to and from the loading nest 316 which can store or support a loaded multi-plate frame 240. Any of the foregoing steps or processes can be performed simultaneously with any combination of other processes being performed by the processing system 300, i.e., while the processing system 300 performs other operations such as, but not limited to, any combination of the following steps: mixing the test substance using the stir plates, mixing the test substances with pipette mixing, dilution of the test substance, serial dilution of the test substance, processing the test plates by adding or pipetting one or more test substance to the test plates, loading up new test plates on the loading nest 316, and/or any other desired test plate operations. The processing system 300 is configured so that the processing system 300 can perform any combination of the steps or operations described in this disclosure simultaneously with other steps or operations described in this disclosure to improve the processing efficiency of the system 300.

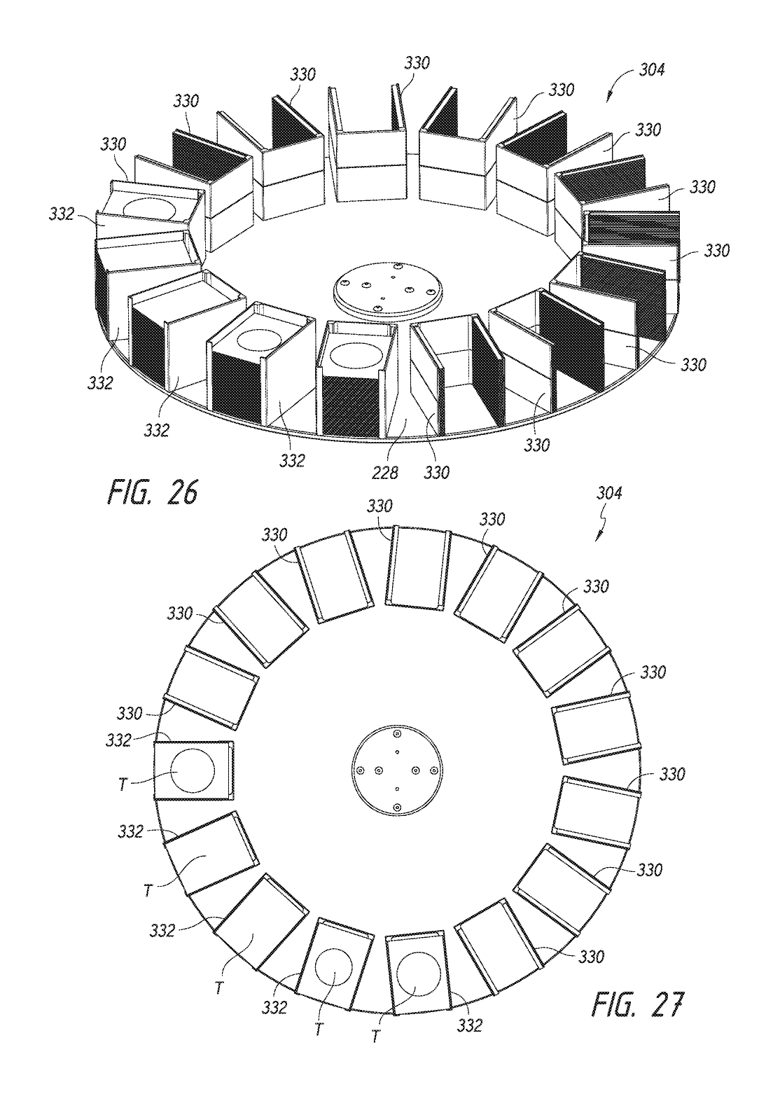

In any embodiments disclosed herein, the test plate storage device 304 can be configured to support or house a plurality of test plates, including the PETRIFILM™ test plates manufactured by 3M™ and/or any other types of test plates disclosed herein or known in the field, whether currently known or developed after the date of this disclosure. One embodiment of the test plate storage device 304 is shown in FIGS. 24-25, and in FIGS. 26-27. Other embodiments of the test plate storage device 304 can have a different shape or configuration. For example and without limitation, other embodiments of the storage device 304 can have linear actuator devices or systems such that the storage modules are arranged in a linear array or pattern instead of a circular array or pattern like in the illustrated embodiment. Additionally, other embodiments of the storage device 304 can be configured to have or provide for additional vertical positions for the storage modules, or otherwise.

With reference to FIG. 26, the test plate storage device 304 is arranged in a circular array. The test plate storage device 304 is configured to rotate about a centerline axis through the approximate center of the base 328. Any suitable controllable motor can be used to rotate the base about the centerline axis, including a stepper motor, a servo driven motor with an encoder feature, etc. The test plate storage device 304 can be configured so that a user can control the orientation or indexing of the base 328 and all of the storage modules 330, 332 (also referred to herein as test plate hotels) supported by the base 328 of the test plate storage device 304 relative to any desired position on the deck 302 or relative to any of the other components of the test plate processing system 300. For example, a motor with an encoder, one or more sensors, and/or other indexing components can be used to control the indexing of the base 328 of the storage device 304 so that the test plate handling device 240 can accurately remove and insert test plates from any one of the plurality of storage modules 330, 332 supported by the base 328.

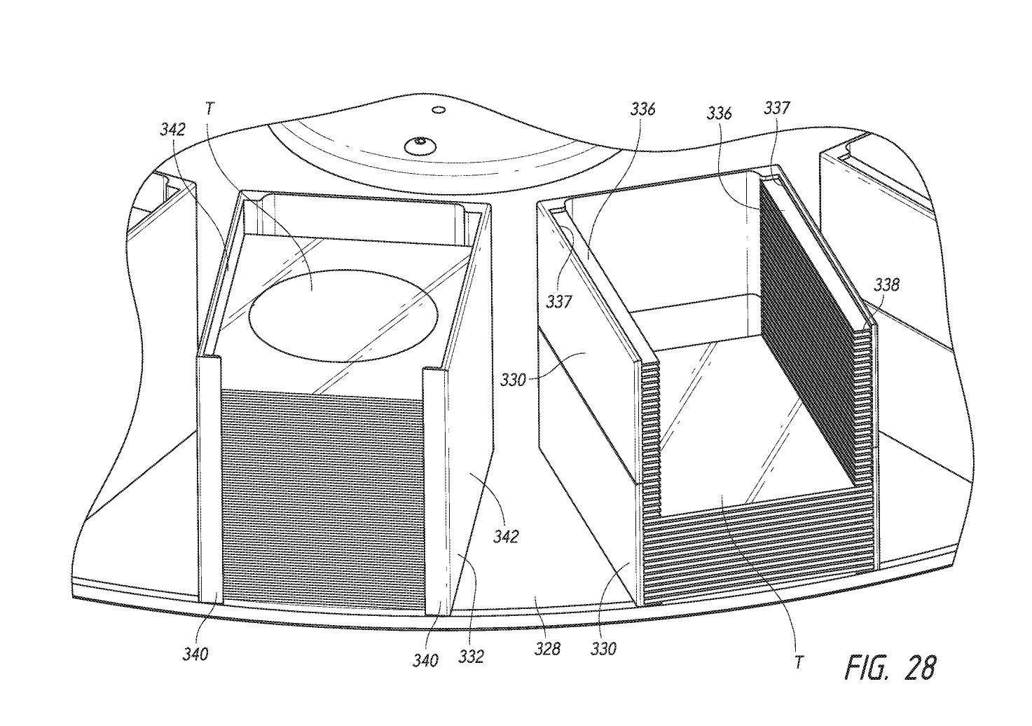

In any embodiments disclosed herein, any of the storage modules 330, 332 can be slotted, such as in storage module 330, or unslotted, such as in storage module 332. For example and without limitation, any of the storage modules 330 can have continuous, unslotted side walls. FIG. 28 is an enlarged view of a portion of the embodiment of the test plate storage system shown in FIG. 26, showing the features of the slotted and unslotted storage modules 330, 332 in more detail. With reference to FIG. 26, the slotted storage module 330 can have a plurality of equally spaced ledges or support tabs 336 that project inwardly away from the inside walls 337 of the storage modules 330. Between each of the support ledges is a space 338 into which a test plate can be advanced or inserted. Any test plates can be inserted into the spaces 338. For example, without limitation, processed test plates (test plates that have already received the sample substance therein) can be stored in the slotted storage module 330, thereby resulting in a gap or space between each of the processed test plates. The gaps can beneficially permit each of the test plates in the storage module 330 additional exposure to heat and/or warm air when the storage module is positioned in an incubator, thereby improving the probability that each of the processed test plates will be evenly and consistently heated when the storage module is in an incubator. In any embodiments, the thickness of the support tabs 336 can be approximately 0.030 in, or from approximately 0.015 inch to approximately 0.060 inches or more. In any embodiments, the thickness can be approximately the same as the thickness of the test plate, or from approximately the same thickness as the test plate to approximately 200% of the thickness of the test plate, or greater, depending on the amount of space desired between each test plate when supported in the slotted storage module 330.

Any storage device 304 embodiments can have one or more marker plates that can be used to indicate that a storage module has twenty test plates loaded in it. The marker plate can be used to signal to a user that a storage module has twenty modules loaded therein, thereby eliminating a need for the user to count the number of test plates in a storage module. Any processing systems 300 can be configured to automatically load the marker plate in any storage module that has twenty test plates therein.

The storage modules 332 can be configured to be open on the inside walls thereof to permit the test plates to be inserted vertically into the storage module. In this arrangement, a stack or plurality of test plates can be inserted by an operator or with robotic equipment into the storage module simultaneously, making the loading of the storage plates into the unslotted storage modules 332 quicker and more efficient. This configuration can be best suited for storing new, unprocessed test plates. The storage module 332 can have, but is not required to have, a tab or projection 340 extending inwardly from each of the sidewalls 342 two limit or restrain the movements of the test plate T in the horizontal or lengthwise direction, so that the test plates T do not inadvertently slide out of the storage modules 332 during operation of the processing system 300 or otherwise. The projections 340, or partial end walls, as they can be referred to, can also help maintain the position of the test plates more accurately in the horizontal directions to ensure the test plates are accurately located in the storage device 304 so that they are accurately positioned on the transport device 364 when the transport device 364 retrieves the test plates from the storage modules 332.

The storage modules 330, 332 can be any desired height or size, depending on the size of the test plate desired to be stored in the storage module, and/or the number of test plates desired to be stored in the storage module. For example, without limitation, a storage module 332 that is approximately 8.85 inches tall can store approximately 200 or slightly more test plates. A slotted storage module 330 or a stack of two or more slotted storage modules that is approximately equal in height with an unslotted storage module 332 can be configured to store approximately half as many test plates as compared to an unslotted storage module. For example, a slotted storage module 330 or a stack of two or more slotted storage modules that is approximately 8.85 inches tall can store approximately 100 or slightly more test plates.

In some embodiments, a stack of 3 or more, or 4 or more, or 5 or more slotted storage modules can be positioned at each radial position about the base 328 of the storage system. In some embodiments, the storage device 304 can have the capacity to store from 20 to 100, or from approximately 20 to approximately 50 processed test plates at each position on the base 328, or from approximately 40 to approximately 200 unprocessed test plates at each position on the base 328.

In any embodiments, the storage device can be configured to store an equal number of processed and unprocessed test plates. In any embodiments, though not required, the illustrated embodiment of the storage device can have two times as many slotted storage modules as unslotted storage modules. For example and without limitation, in any embodiments, as in the illustrated embodiment, the storage device can have 10 slotted storage modules 330 and 5 unslotted storage modules 332, or 20 double stacked slotted storage modules 330 and 5 unslotted storage modules 332, as illustrated. In the illustrated embodiment, the base can have a diameter of approximately 26.4 inches, or from approximately 12 inches to approximately 30 inches or more. The size of the base can be adjusted depending on the desired number of storage modules and/or the space available for the storage module on the deck.

Additionally, as shown in FIG. 28, the storage modules 330, 332 can be designed to have features such as indexing pins, bosses, projections, recesses, and/or other nesting and/or locating features to permit the storage modules to be stacked one on top of another and also to be accurately positioned on the base or deck. Additionally, each of the storage modules 330, 332 can be rigid enough such that a robotic arm or other grasping mechanism can grasp one or more of the storage modules on the outside walls thereof to move the entire storage module(s) with the processed and/or unprocessed test plates located within the storage module(s).

With reference to FIG. 24, more details about the test plate handling system 306 will now be described. The test plate handling system 306 can be supported by the deck 302 of the test plate processing system 300. In any embodiments, though not required, the test plate handling system 306 can have a support structure 350 that supports the test plate handling system 306 above portions of the deck 302. For example, as illustrated, the support structure 350 can have any number of vertical support members 352 (four being shown) and horizontal support members 354 (four being shown, though the horizontal support member 354b can have a portion removed therefrom to permit the free movement of the test plate transport device 364) coupled together form a frame structure to support the test plate handling system 306. In any embodiments, the horizontal support members 354b, 354c can be configured to terminate before reaching the opposite vertical support member 354 so as to leave a space in the horizontal support member that permits the movement of the test plate transport device 364 to move freely between the multi-plate holder 240 and the storage device 304.

Additionally, in some embodiments, support members 360 can be coupled with the support structure 350 and be sized and configured to support the components of the spreader device 366. One or more brackets 380 can be used to attach or couple the components of the spreader device 366 with the support members 360. In any embodiments, additional support members can be sized, configured, and positioned to support one or more components of the test plate transport device 364, thereby permitting the support structure 350 to support the test plate transport device 364.

Any of the support members of the support structure 350 or otherwise used in the test plate processing system 300 can comprise T-slot extruded aluminum framing system members or any other suitable framing members. The support structure 350 can support any of the components of the test plate handling system 306, including the housing cover 358 and the test plate transport device 364. Additionally, in any embodiments disclosed herein, as will be described in greater detail below, the support structure can also support an automated spreader device 366.

Also, with reference to FIG. 24, a positioning device 367 can be supported directly on the deck 302, as illustrated, or, in other embodiments, by the support structure 350. In the illustrated embodiment, the positioning device 367 can be a linear actuator device (that can be of the type typically known or used in the art) that can controllably move the multi-plate holder 242 between a plurality of predesignated or desired positions as the test plate transport device 364 moves test plates between the storage device 304 and the desired portion 242 of the multi-plate holder 240. The positioning device 367 can move the multi-plate holder 240 into any of a plurality of positions such that either the first test plate holder portion 242a, the second test plate holder portion 242b, or the third test plate holder portion 242c is aligned with the movement path of the test plate transport device 364 or with a lateral center of a test plate T being moved by the test plate transport device 364. In this arrangement, the test plate transport device 364 can position the test plate T in any of the first test plate holder portion 242a, the second test plate holder portion 242b, or the third test plate holder portion 242c of the multi-plate holder 240.

A control system or software controller can be used to control the positioning and sequencing of any of the devices and components of the test plate processing system 300. In any embodiments, each of the motors used in the system can have its own controller to control an operation of the motor. Some motors can have controllers integrated with the motors. A computer used to control the processing system 300 can have one or more software drivers for one or more of the controllers. Any embodiments of the processing systems 300 disclosed herein can have one or more communication hubs such as, but not limited to, the CLEARPATH® Communication Hub (part number SC4-HUB). The communication hub(s) can allow the connection of up to four motors to the computer controller that can be used to control the processing system 300. The computer can be configured to send the necessary or desired automation commands to each motor through the communications hub. Commands for the one or more motors of the processing system 300 can be written and executed in the software driver for the system 300. The CL3-E-1 motor controller sold by NANOTEC® is an example of a motor controller that can be used to control one or more motors of the system 300, with one controller controlling one motor. Software offered by NANOTEC®, such as the NanoJ software, can be used to configure the motors and/or controllers. Additionally, the OpenCM9.04-C is an example of a controller for Dynamixel motors. In any embodiments, the motor controllers can be connected to the main computer or a programmable logic controller.

Additionally, in any embodiments disclosed herein, the multi-plate holder 240 can be configured to support more than three test plates, such as a multi-plate holder that can support four test plates and has four portions therefore, or a multi-plate holder that can support five test plates and has five portions therefore. In some embodiments, the multi-plate holder can even be configured to support more than five test plates, being configured to support any desired number of test plates either in a single row, or into multiple row, multiple column arrangement. The positioning device 367 can be sized and configured to support any size or configuration of the multi-plate holder 240 that is desired to be used in the test plate processing system 300.

The positioning device 367, as mentioned, can be a linear actuator that can controllably position the multi-plate holder 240 or any of the test plates T in the desired position relative to the test plate transport device 364. Additionally, an automated spreader device 366 can be supported or positioned adjacent to the test plate transport device 364 that can, if needed in a system and if included in a test plate processing system (though not required), be used to spread the test substance in the test plates using any one of a plurality of different spreader elements. The spreader device 366 can apply a force in a downward vertical direction that is collinear with a normal axis of the test plate against the test plate to spread and prepare the test substance according to the needs of the particular test being done. In other embodiments, the spreader device 366 can be positioned anywhere on the deck 302 or otherwise.

When the spreader device 366 is included in the system, the positioning device 367 can have a longer length so that positioning system 367 can position the multi-plate holder 240 in the desired position relative to the spreader device. For example without limitation, the positioning device 367 can move the multi-plate holder 240 such that either the first test plate holder portion 242a, the second test plate holder portion 242b, or the third test plate holder portion 242c is approximately lined up with the movement path or lateral centerline of the spreader device 366 so that the spreader device 366 can spread the test substance in the plate T in any of the first test plate holder portion 242a, the second test plate holder portion 242b, or the third test plate holder portion 242c of the multi-plate holder 240 by pressing down against the respective test plate.

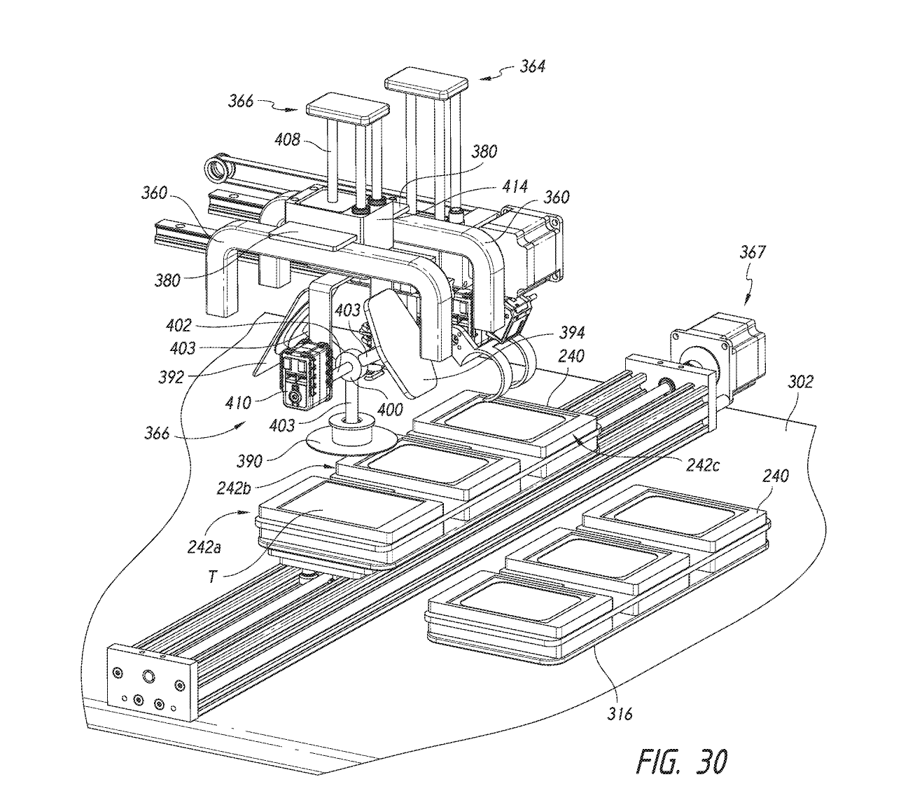

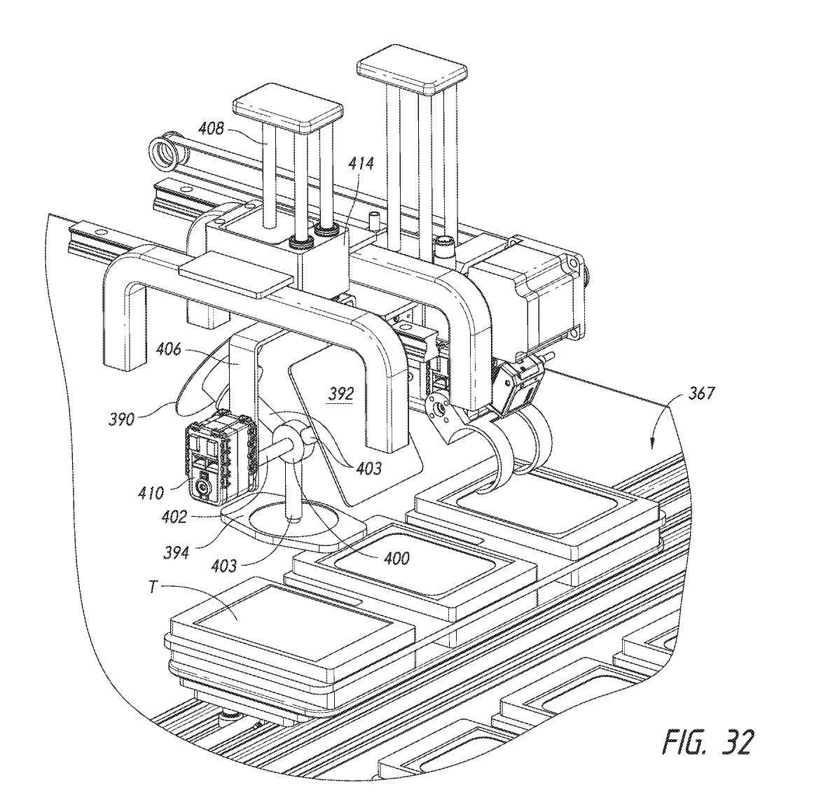

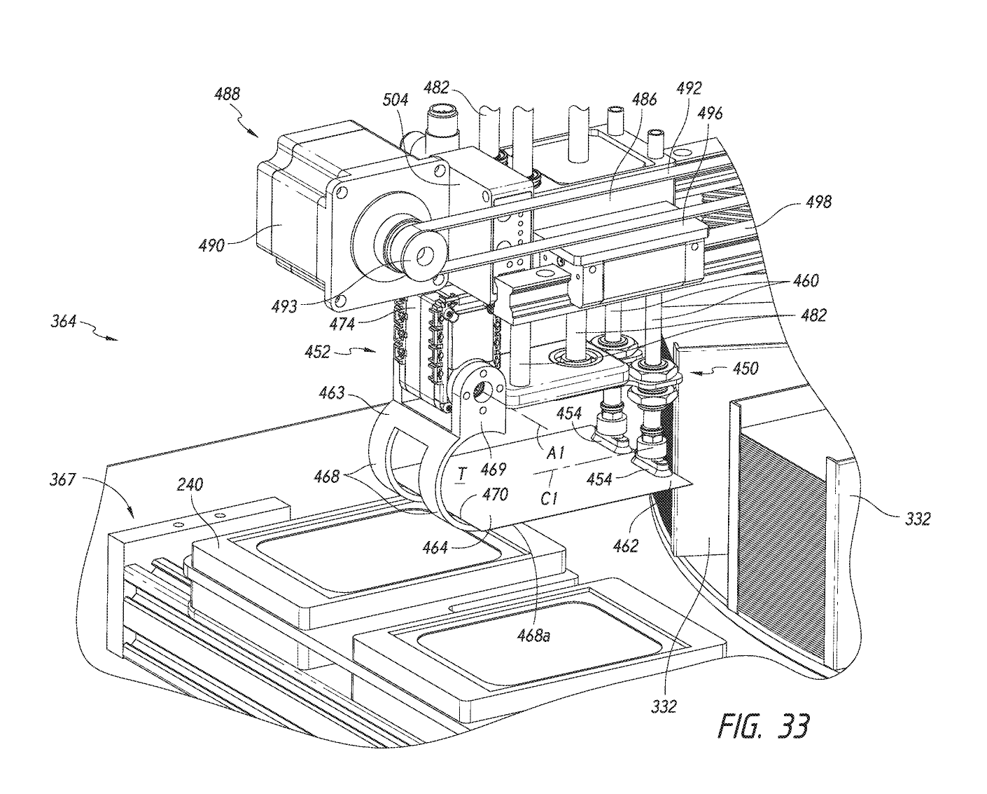

Any embodiments of the spreader device 366 can have a plurality of spreader elements thereon, and any of the spreader elements can be removable or interchangeable for other spreader elements. For example without limitation, with reference to FIG. 30, the spreader device 366 can have a first spreader element 390, a second spreader element 392, and a third spreader element 394 supported by a spreader base 400. A shaft 402 can be coupled with the spreader base 400 to permit the spreader base 400 to controllably rotate about a centerline axis of the shaft 402 such that any one of the desired spreader elements 390, 392, 394 can be positioned in an operable position to apply a downward pressure on the test plate T to spread the test substance on the plating surface P of the test plate T. For example, with reference to FIG. 30, the spreader element 390, which has a circular shape, is positioned in the operable position to apply a downward pressure on the test plate T supported on the first portion 242a of the multi-plate holder 240, as shown in FIG. 30. FIG. 32 shows spreader element 394 in the operable position to apply the downward pressure on the test plate T.

The spreader elements in any embodiments herein can have any desired shape, thickness, stiffness, material, or otherwise suitable for such spreading operations. Further, any of the spreader elements can comprise any of the sizes, shapes, materials, components or parts, or other features of or similar to the PETRIFILM™ spreaders manufactured by 3M.

Additionally, as mentioned, any of the spreader elements 390, 392, 394 can be removed from the spreader base 400 and interchanged with any other desired spreader element. Additionally, in any embodiments disclosed herein, the spreader device 366 can have any number of supports for any number of spreader elements, including two spreader elements, four spreader elements, five spreader elements or more. Each of the spreader elements 390, 392, 394 can be coupled with the spreader base 400 and any suitable fashion and using any suitable support structure, including, without limitation, the shafts 403 shown in the figures.

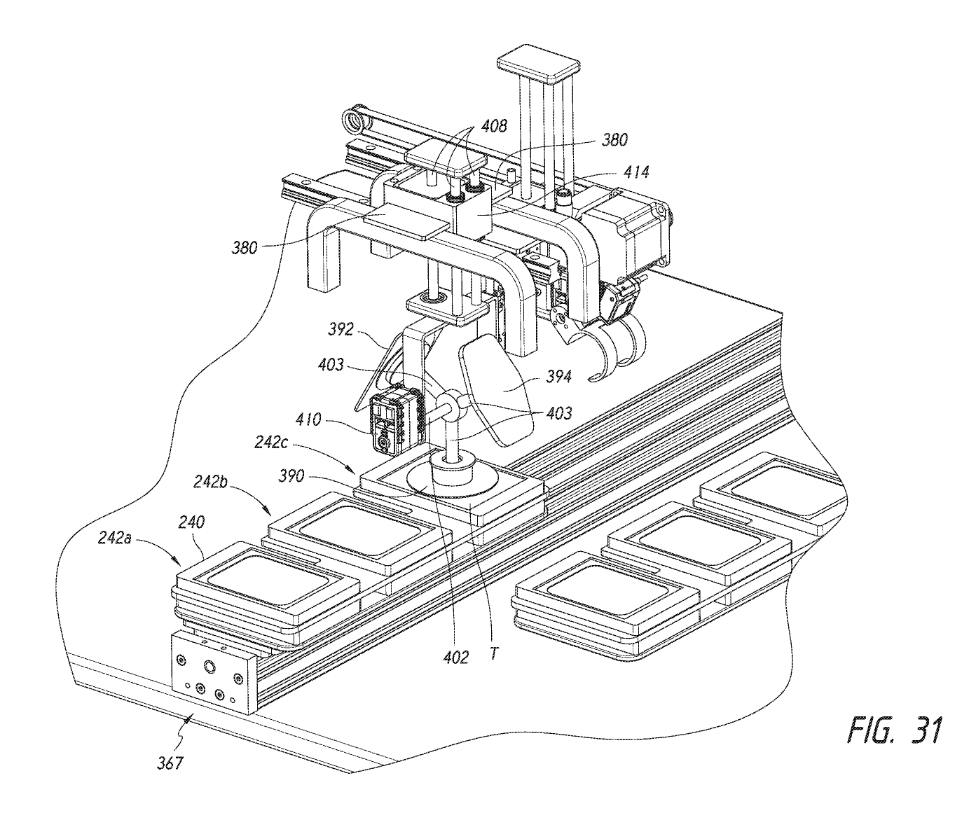

With reference to FIG. 31, the positioning device 367 can move the multi-plate holder 240 along the length of the positioning device 367 so that the desired portion 242 of the multi-plate holder 240 is in the desired position in approximate alignment with the spreader device 367 and the desired spreader element 390, 392, 394. As shown in FIG. 31, the multi-plate holder 240 has been moved along the positioning device 367 such that the third portion 242c of the multi-plate holder 240 has been positioned in alignment with the circular spreader element 390.

A bracket 406 can be configured to support an end portion of the shaft 402. The bracket 406 can also be coupled with end portions of two or more shafts 408 (three being shown) to permit the bracket 406 to move up and down in the vertical direction when the shafts 408 are moved up and down in the vertical direction. A controllable motor system 410 can be coupled with or supported by the bracket 406 and can be configured to rotate the shaft 402 into the desired radial position such that the desired spreader elements is in the operable position. The controllable motor system 410 can control the indexing of the shaft 402 to thereby control the positioning of the shaft 402 and the spreader body 400. A main control system or software system can be used to control the operation of the controllable motor system 410.

Additionally, a controllable linear actuator 414 can be used to move the shafts 408 up and down in the vertical direction. The linear actuator 414 can comprise any suitable features or components, including the features and components of a voice coil actuator, a vacuum driven actuator, piezo ultrasonic actuator, a linear bearing actuator, and/or any other suitable type linear actuators or computer-controlled positioners. The linear actuator 414 can be supported and/or coupled to the support structure 350 using one or more brackets or support members, such as the two brackets 380 shown in the figures.

The motor can be supported in a stationary position, while the leadscrew can move up and down. One characteristic of this arrangement is that, when the leadscrew goes up or down, the entire sub-assembly with the spreader elements also goes up or down, so that the spreader elements can move up or down in a controllable fashion. The linear actuator 414 can be any of a variety of configurations, including, without limitation, as described below. In some embodiments, the linear actuator 414 can comprise a non-captive motor that contains a preinstalled leadscrew that moves from within the motor. One example of a non-captive stepper motor is the NANOTEC LA42-linear actuator series. Some embodiments can include an encoder so that the motor can keep track of its location and movement. The linear actuator 414 can also comprise a linear bearing and a steel shaft. One or more flange ball bearings can support one or more of the end portions of the shaft, and an aluminum block can be used to support the motor and linear bearings. IN any embodiments, the linear actuator 414 can comprise a screw drive or threaded shaft. Additionally, any embodiments of the linear actuator can comprise one or more components, parts, details, or other features of the Z-Axis components by TECAN™.

FIG. 33 is an enlarged view of a portion of the embodiment of the test plate processing system 300 shown in FIG. 24, showing the embodiment of the test plate transport device 364 of the processing system embodiment shown in FIG. 24. In any embodiments disclosed herein, the test plate transport device 364 can have a suction element 450 that can be used to pick up or grasp any desired test plate T. In some embodiments, the suction element 450 can have a single suction manifold 452. In some embodiments, the suction element 450 can have a first suction manifold 454 and a second suction manifold 454 positioned side-by-side. The first and second suction manifolds can be positioned symmetrical about a lateral centerline C1 of the test plate T. The first and second suction manifolds can be configured to simultaneously contact a top surface of a test plate T and then simultaneously exert a suction force on the top surface of the test plate T so as to grasp or engage the test plate T. Additionally, suction can be applied to the one or more suction manifolds before the suction manifolds make contact with the test plate, or after the one or more suction manifolds make contact with the test plate.

To be clear, in any embodiments disclosed herein, a single suction manifold can be used, such as suction manifold 454, that can be positioned along a lateral centerline of the test plate T. Any of the suction manifolds disclosed herein can have an oval shape or oval shape with parallel sides, similar to what is shown in FIG. 33, and can have any desired length or width. For example without limitation, in any embodiments disclosed herein, the suction manifold can have a length that is approximately 20% to approximately 40% of the width of the test plate that the suction manifold is being used with, or from approximately 10% to approximately of the width of the test plate that the suction manifold is being used. Additionally, any of the suction manifolds can have a circular shape, square shape, rectangular shape, or otherwise.

Each suction manifold that is used can be coupled with suction tubing 460 that can provide a source of reduced pressure or suction pressure to the suction manifold. Additionally, in any embodiments, a single suction tubing 460 can be used to provide a source of reduced pressure or suction to one, two, three, or more suction manifolds used to lift the test plate T. Where a single suction tubing 460 is used to supply reduced pressure or suction to multiple manifolds, splitters can be used to deliver the suction to the multiple manifolds. However, in any embodiments disclosed herein, two redundant suction tubings 460 can be used, with each supplying and independent source of reduced pressure to each manifold such that a hole or rupture in one of the lengths of tubing will not substantially affect the system's ability to provide suction or reduced pressure to the another one of the manifolds.

A control system can be used to control the level of pressure supplied to the suction manifold or manifolds, and can control the application and release of the reduced pressure or suction to the suction manifold or manifolds. In this configuration, the one or more suction manifolds 454, 456 can be lowered into contact with the test plate T at a first end 462 of the test plate T (which can be either end of the test plate, including the end where the cover of the test plate is affixed with the plating surface of the test plate), and suction can be applied to the manifolds 454. Thereafter, with suction still applied to the manifolds, the suction manifolds 454 can be lifted in a vertical direction so as to also lift the test plate T in the vertical direction away from either the test plates positioned below the lifted test plate that are in a storage module, away from the multi-plate holder, or otherwise. Thereafter, the manifolds can be moved in the horizontal direction to translate or transport the test plate T to any desired location in the system 300.

Additionally, though not required and although only included in some arrangements of the test plate processing system 300, some embodiments of the test plate transport device 364 can have an optional lift mechanism 452 that can have an engagement element 463 that can engage and lift a second end portion 464 of a test plate. In some embodiments, though not required, the engagement element 463 can be rotatable about an axis of rotation A1 so that one or more elongations 468 extending away from the body portion 469 of the engagement element 463 can be advanced past a distal end portion 470 of the test plate. In this configuration, a distal end portion(s) 468a of the one or more elongations 468 can be positioned underneath the distal end portion 464 of the test plate. Thereafter, when the elongations 468 are lifted in the vertical direction, the lift mechanism 452 can lift the distal end portion of the test plate in the vertical direction.

In the illustrated embodiment, the engagement element 463 is configured to have two elongations 468 extending therefrom, each of the elongations having a circular or arcuate shape having a centerline that is parallel with the axis of rotation A1. However, in any other embodiments, the elongations can be flat or linear shape, can have one, three or more elongations, or can have any other shape or configuration desired. Additionally, the optional lift mechanism 452 can be configured such that the elongations 468 move in a horizontal or even an upwardly angled direction, with or without rotation, so that the distal end portions of the elongations are advanced underneath and past the distal end portion of the test plate along a linear movement path. A controllable motor module 474 can be used to control the movement in one or more directions, or to control the rotational movement, of the engagement element 463. As mentioned, though not required, the optional lift mechanism 452 can work in conjunction with the suction element 450 to lift a second end portion of the test plate from any desired location, hold the test plate while the test plate is being moved by the transport device 364, and to lower and release the test plate in the desired location or position in the system 300.

In any embodiments disclosed herein, the suction element 450 and/or the optional lift mechanism 452 can be supported by a bracket or support member 480 that is coupled with one or more shafts 482 (three being shown). The shafts 482 can be moved up and down using a controllable linear actuator 486 such that the suction element 450 or the suction element 450 and the lifting mechanism 452 can be controllably moved up and down in the vertical direction. The linear actuator 486 can comprise any suitable features or components, including the features and components of a voice coil actuator, a vacuum driven actuator, piezo ultrasonic actuator, a linear bearing actuator, and/or any other suitable type linear actuators or computer-controlled positioners. The linear actuator 486 can be supported and/or coupled to the support structure 350 by being coupled with one or more brackets or slide mechanisms 496 (also referred to as a carriage or a ball bearing carriage) that can translate along the slide rails 498. The linear actuator can be any of a variety of configurations. One nonlimiting example of a linear actuator that can be configured to work with the test plate transport device 304 is a recirculating ball bearing guide and ball screw drive such as the FESTO™ EGSK-26-200-6P, referred to as the Festo Electrical Slide.

In some embodiments, the suction element can initiate the lifting of the test plate in the upward direction. Thereafter, once the test plate is lifted high enough to advance the elongations under the test plate without engaging or hitting an adjacent test plate, the elongations can be advanced past the distal end of the test plate and in contact with a bottom surface of the test plate. Conversely, in some embodiments, when a test plate is being set down or deposited in the desired location, the elongations can be retracted while the test plate is still a predetermined distance (which can be twice the thickness of the elongations, or three times the thickness of the elongations, or any other suitable distance) above the surface on which the test plate will be deposited or set down so that the elongations can be removed without contacting the surface or object adjacent to or below the test plate.

Additionally, any embodiments of the test plate transport device 364 can have a linear displacement mechanism 488 to move the bracket 480 and the suction element 450, or the suction element 450 and the optional lifting mechanism 452, in the horizontal direction so that the suction element 450 and/or the lifting element 452 can be moved between the storage device 304 and any other components, such as the multi-plate holder 240 positioned on the positioning device 367. Any suitable linear slide mechanism can be used to move the test plate transport device 364 in the horizontal direction. In the illustrated embodiment, the linear displacement mechanism 488 can have a controllable motor 490 (which can be a servo driven motor, such as but not limited to the TEKNIC CLEARPATH® CPM-SCSK-2321S-RLNB servo motor) configured to drive a belt 492 that is configured to travel around a first pulley 493 supported by a shaft coupled with the motor 490 and a second pulley 494 supported by a bracket (not shown) that can be coupled with or be supported by the support structure 350. The belt can be coupled with a first bracket or slide mechanism 496 such that, as the belt translates in the horizontal direction, the first slide mechanism 496 will simultaneously and equally translate in the same horizontal direction along the rail 498 which can be coupled with or supported by the support structure 350. The first bracket or slide mechanism 496 can be coupled with or attached to, or monolithically formed with, a housing or case of the controllable linear actuator 486. Additionally, a second bracket a slide mechanism 496 can also be attached to or coupled with the housing or case of a controllable linear actuator 486 to further stabilize the movement of the actuator in the horizontal direction. In any other embodiments, a linear slide device having a leadscrew can be used to move the bracket 480 and the suction element 450, and/or other components of the test plate transport device 364.

Further, in any embodiments disclosed herein, the test plate transport device 364 can further support a distance sensing or measuring mechanism 504, which can be a laser distance sensor that can determine a distance between the suction elements 450 and/or the engagement element 452 and the test plate to be engaged by the transport device 364 so that the transport device 364 is lowered to the correct height to retrieve the test plate. The information obtained by the distance sensing mechanism can be fed into the controller or used to control the controller to move to the correct height relative to the target test plate. For example and without limitation, a MICROTRAK™ 3, a MICROTRAK™ 4, or an ACUITY AR500 laser measurement or displacement sensor can be used. Additionally, sensor switches or limit switches can also be used to limit the downward movement of the transport device 364 and stop the transport device 364 in the correct location or distance from the target test plate.

Furthermore, in any embodiments, the test plate transport device 364 can further support a bar code sensor that can read a bar code on the target test plate. By using the bar code reader and having bar codes on the test plates, the processing system can begin simultaneous operations (such as preparing the test substances, test substance mixing, and other processing operations) necessary for the target test plate.

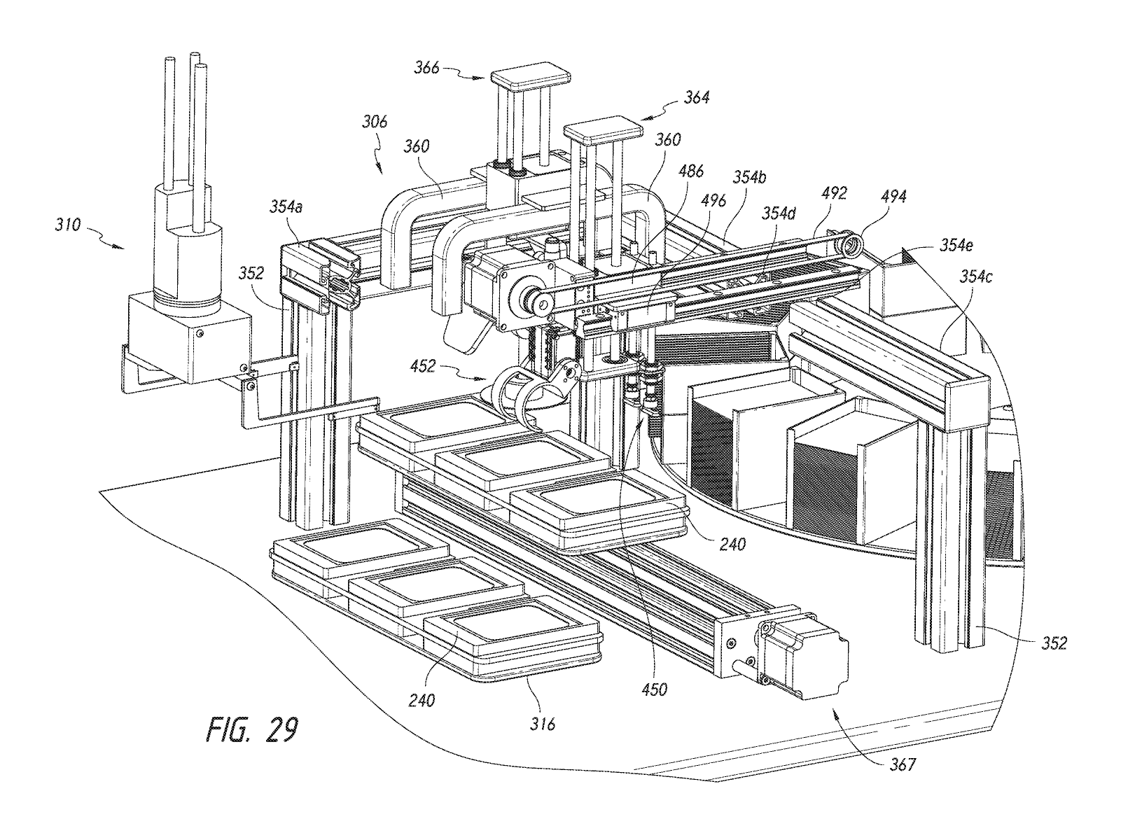

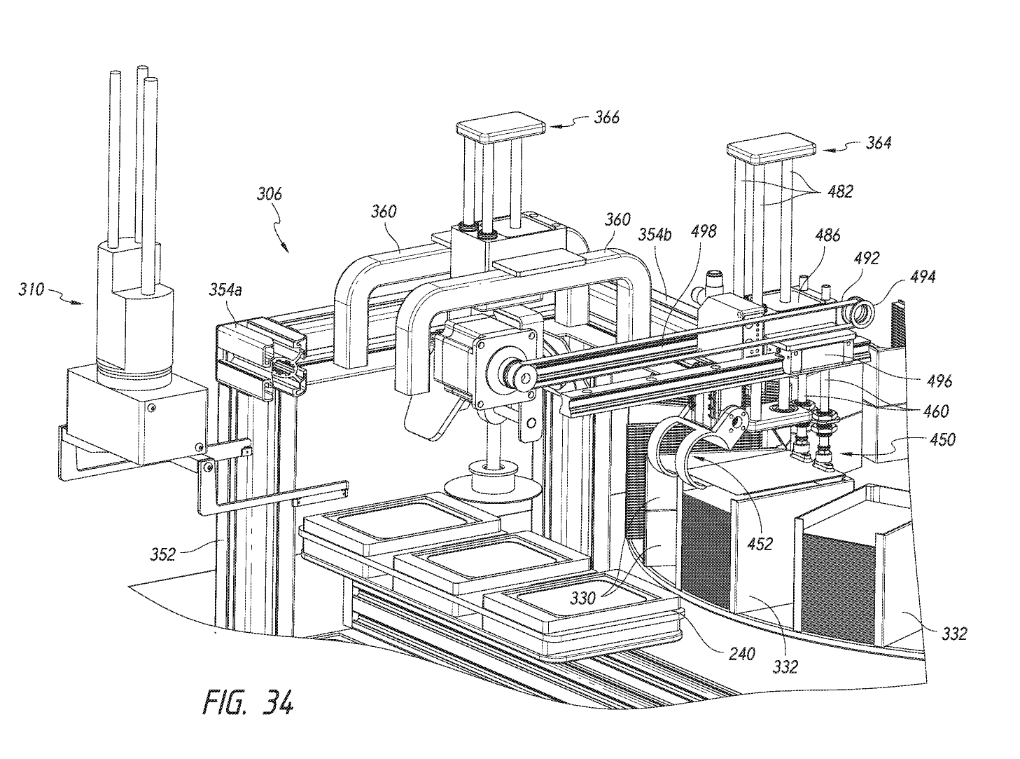

FIG. 34 is an enlarged view of a portion of the embodiment of the test plate processing system 300 shown in FIG. 24, showing the embodiment of the test plate transport device 364 lifting a test plate T. Just as in FIG. 29, the multi-plate holders 240 shown in FIG. 34 have not been loaded with any test plates T. FIG. 34 shows the transport device 364 undertaking one or more steps to load a test plate T in the multi-plate holder 240. As shown in FIG. 34, the storage device 304 has moved to the appropriate indexing position to permit the desired test plate T to be removed from the desired storage module 332 by the test plate transport device 364. Also, the suction element 450 has engaged with the test plate T and has lifted the test plate in a vertical direction a short distance. At this point, if the lift mechanism 452 is part of the configuration, the lift mechanism 452 can be used, but is not required to be used, to support the second distal end portion of the test plate T. The one or more elongations 468 can be advanced so that the distal end portion of the elongations 468 are positioned under the distal end portion of the lifted test plate.

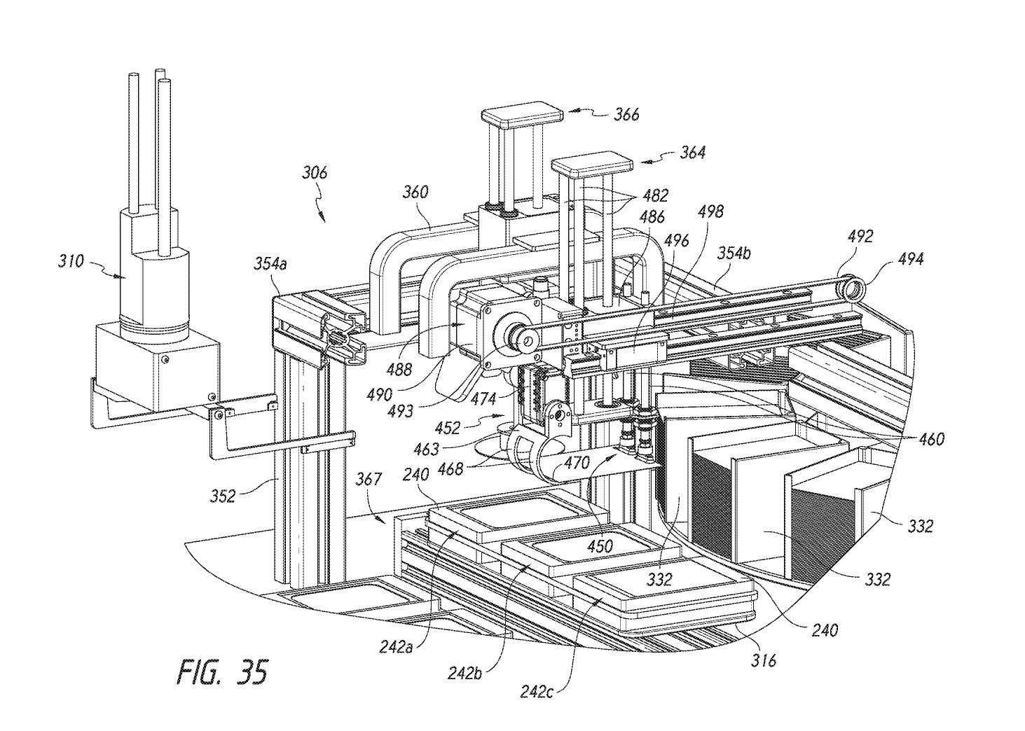

FIG. 34 shows this arrangement before the elongations 468 have been advanced underneath the distal end portion of the lifted test plate. FIG. 35 is an enlarged view of a portion of the embodiment of the test plate processing system 300 shown in FIG. 24, showing the embodiment of the test plate transport device 364 moving the test plate. FIG. 35 shows the elongations 468 having been advanced underneath a distal end portion of the test plate and the test plate transport device 364 has moved partially away from the storage device 306 toward the multi-plate holder 240.

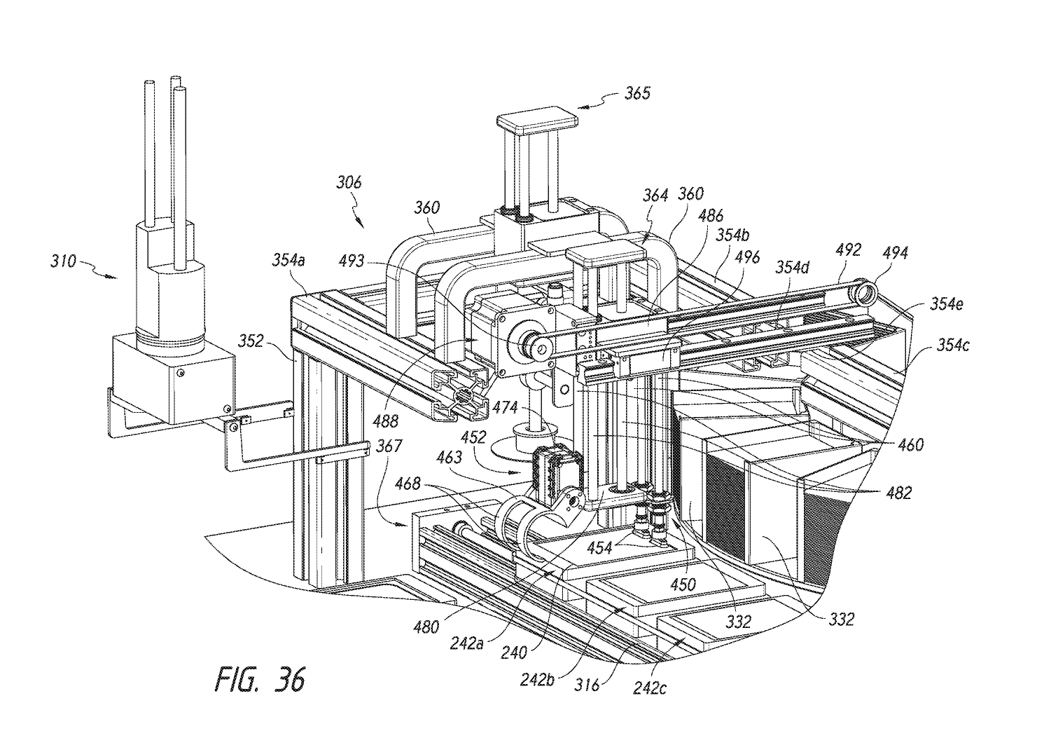

FIG. 36 is an enlarged view of a portion of the embodiment of the test plate processing system shown in FIG. 24, showing the embodiment of the test plate transport device 364 depositing a test plate on a predetermined portion of the multi-plate holder 240 embodiment shown in FIG. 24. As can be seen in FIG. 36, the lifting mechanism 452 has been retracted and the suction element 450 is still in contact with the test plate, with the test plate being positioned or secured in contact with the desired portion of the multi-plate holder 240. Thereafter, the suction can be released from the suction element and the transport device 364 can be lifted in the vertical direction and moved to the next desired position. Test plates can be positioned on all of the portions multi-plate holder 240 in this manner, after the positioning device 367 has moved the multi-plate holder 240 in the desired position so that the desired portion 242 of the multi-plate holder 240 is laterally aligned with the test plate transport device 364 so that a test plate can be positioned in each of the portions of the multi-plate holder 240. For example, as shown in FIG. 35, the positioning device 367 has aligned the second portion 242b of the multi-plate holder 240 in alignment with the test plate transport device 364. FIG. 36 shows the test plate transport device 364 depositing a test plate T on the first portion 242a of the multi-plate holder 240, after the positioning device 367 has moved the multi-plate holder 240 so that the first portion 242a of the multi-plate holder 240 is generally in alignment with the test plate transport device 364. The positioning device 367 can also move the multi-plate holder 240 in an optimal position for the robotic arm 310 to engage the multi-plate holder 240. The test plate transport device 364 can move so that it does not obstruct the movement or processing by other components or devices of the system 300, including the robotic arm 310. Again, in any embodiments disclosed herein, any combination of the steps or movements described herein can be performed simultaneously to improve processing efficiency.

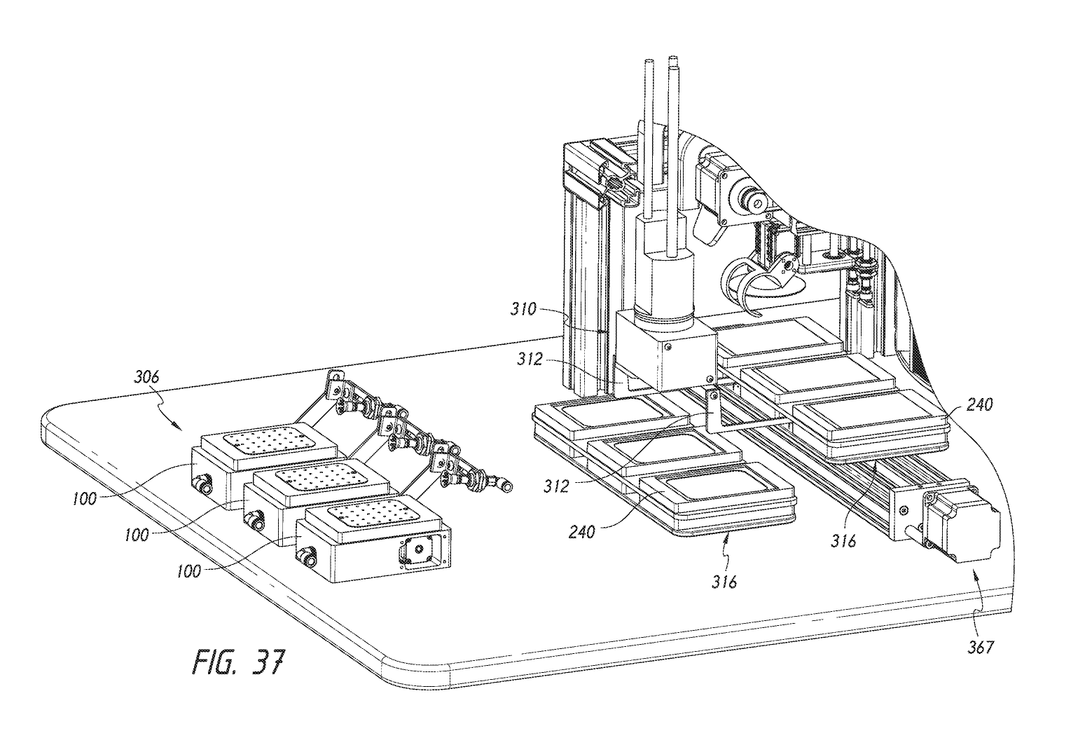

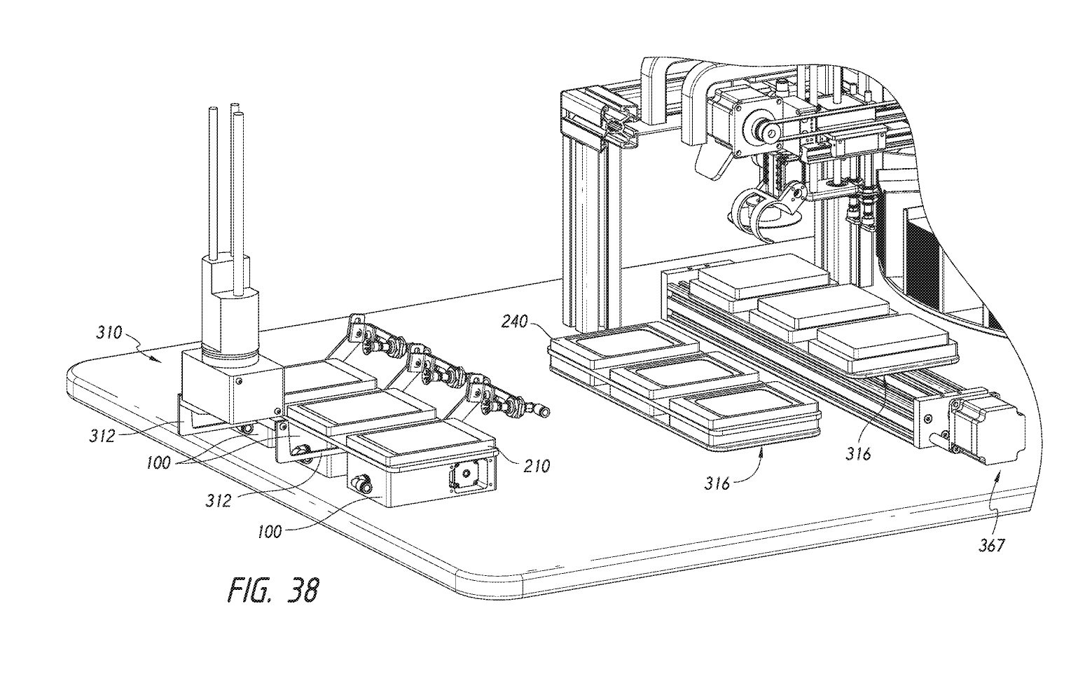

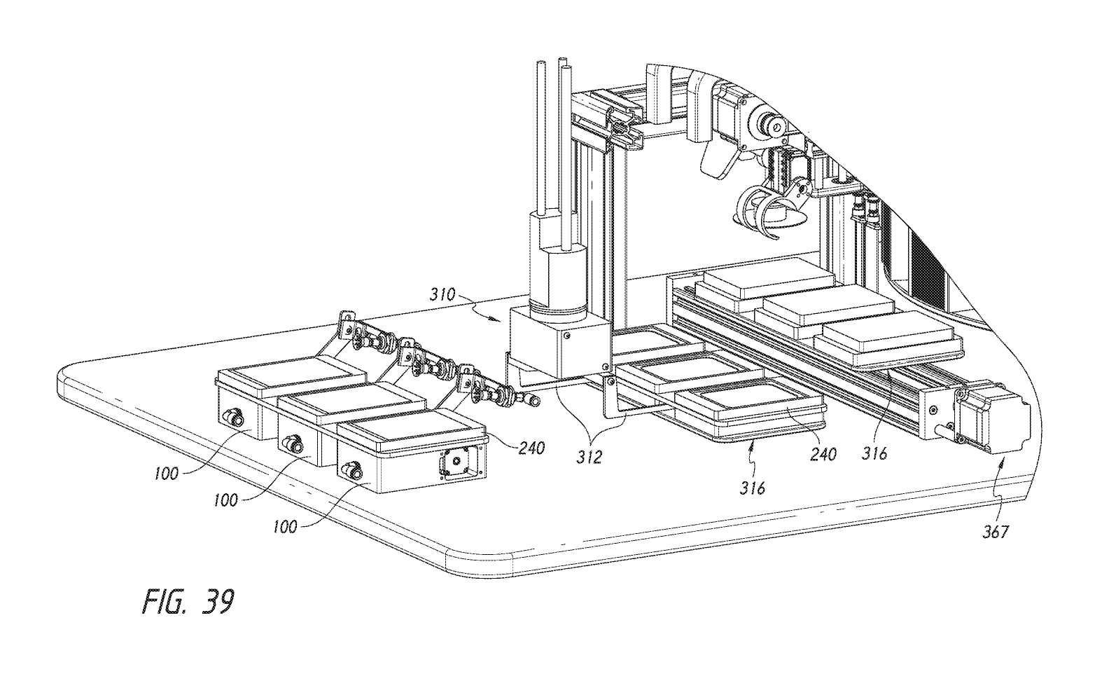

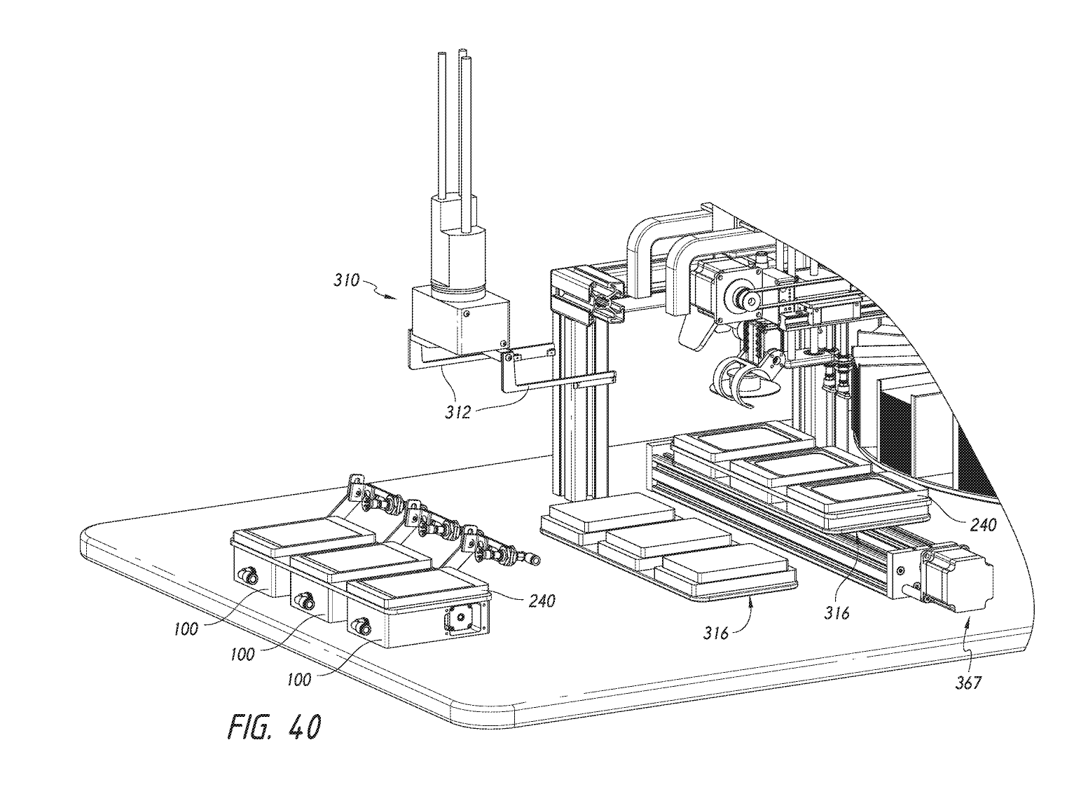

FIG. 37 is an enlarged view of a portion of the embodiment of the test plate processing system 300 shown in FIG. 24, showing the robotic arm 310 engaging the multi-plate holder 240 (having three test plates T supported thereon), preparing to move the multi-plate holder 240 away from the positioning device 367. The robotic arm 310 can thereafter move the multi-plate holder 240 into the desired position on the array of processing devices 100, as shown in FIG. 38. Note that the processing devices 100 can be positioned in any desired location, on or adjacent to the deck 302, or otherwise. Thereafter, the processing devices 100 can process each of the test plates so that the desired test substance is properly applied to each of the test plates. From that position, with reference to FIGS. 39-40, in some embodiments, the robotic arm 310 can engage the multi-plate holder 240 that is positioned on the nesting device 316, and move the multi-plate holder to the positioning device 367 where the test plate transport device 364 can load more test plates onto the multi-plate holder 240.

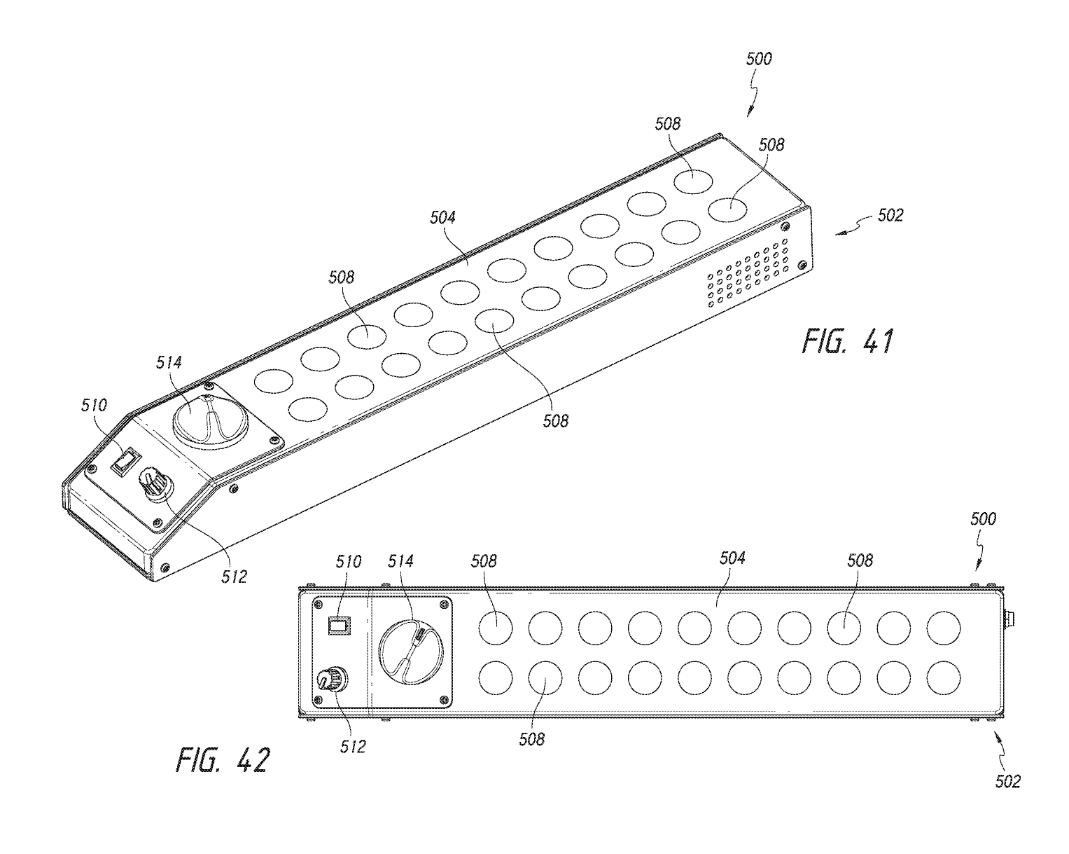

FIGS. 41 and 42 are an isometric view and a top view of an embodiment of a stirring device 500 that can be used independently, or with any of the test plate processing system embodiments disclosed herein, including, without limitation, test plate processing system 300. For example and without limitation, any embodiment of the stirring device 500 can be supported by and/or coupled with the deck of the processing system 300 so that the stirring device can be used to stir any test samples that need to be stirred before processing the test samples.

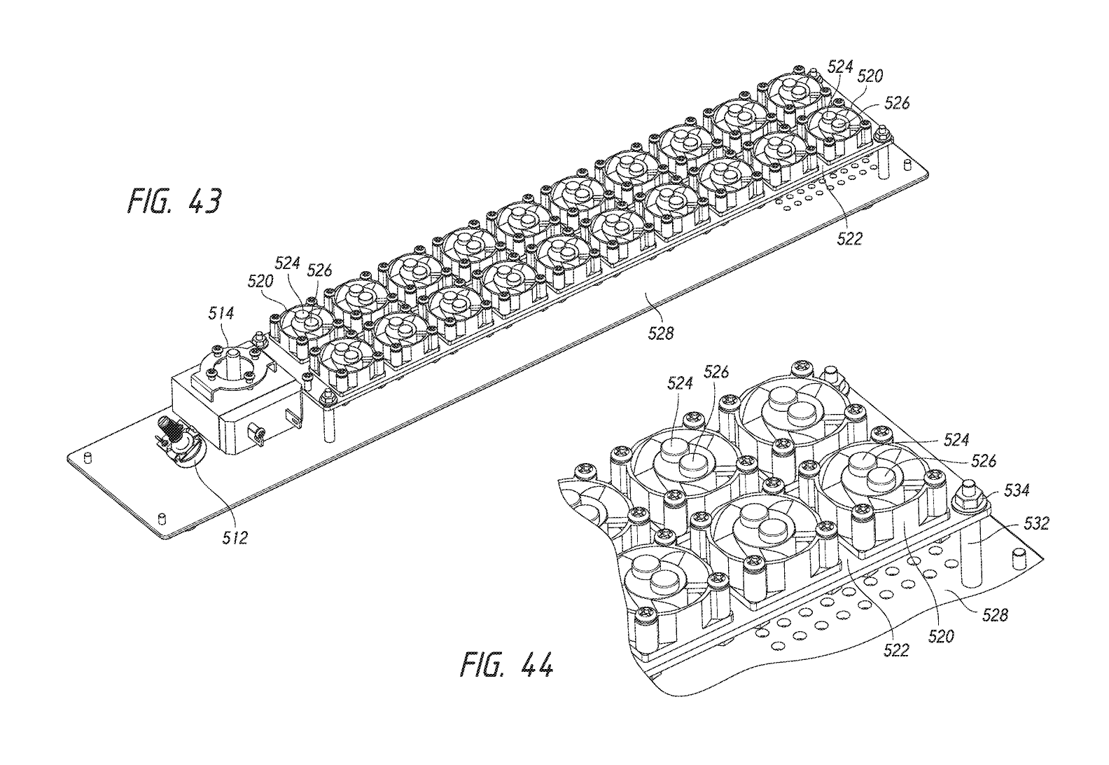

FIG. 43 shows the embodiment of the stirring device 500 shown in FIG. 41, with the housing or cover 502 removed for clarity. FIG. 44 is an enlargement of a portion of the view of the stirring device 500 shown in FIG. 41. In any processing system embodiments disclosed herein, the stirring device 500 can have magnetic stirring elements coupled with or integrated with a rotating element configured to rotate a stirring bar positioned inside of the test container.

With reference to FIGS. 41-44, any embodiments of the stirring device 500 can have a housing or case 502, a top surface 504 having a plurality of location marks 508 (twenty being shown), and a plurality of individually controllable stirring elements 520 (twenty being shown). The stirring elements 520 can be aligned with each of the location marks 508 on the top surface. The location marks are optional in any embodiment, and not required. When a test container is positioned adjacent to the top surface 504 of the stirring device and in alignment with any of the stirring elements 520, the stirring element 520 can be activated so as to cause a stirring bar (not shown) positioned within the test container to rotate in accordance with a rotation of the stirring element 520 of the stirring device 500 so as to mix the test substance within the test container. Any suitable type, size, shape or configuration of a stir bar can be used. The stir bar can be coated with an inert material, such as polytetrafluoroethylene, or other inert plastic material or otherwise. In any embodiments, a stirring bar placed in the test container (containing a liquid or solution) can be rotated by the rotating magnetic field that can be generated by each stirring element 520.

In any embodiments, the stirring elements 520 can comprise a DC fan or other rotating device. An example of a fan that can be used with any embodiments of the stirring device disclosed herein is a 12 volt DC fan being approximately 25 mm by 25 mm by 10 mm in depth or thickness. However, the size and configuration of the fans can be adjusted depending on the size of the test containers to be used with the stirring device 500. A pair of magnets 524, 526 can be coupled with (such as, without limitation, glued or otherwise adhered to) the fan or rotating device to spin the stirring rod. The two magnets 524, 526 can be configured or positioned so as to have opposite polarity on the upward facing surfaces there, so as to attract an end portion of the stir bar having opposite polarity. In any embodiments disclosed herein, the magnets 524, 526 can comprise neodymiun and/or other magnetic materials. Each rotating element 520 can support a magnet or magnets (two being shown). Another possible configuration can have one rectangular magnet per rotating element 520. The rotating element 520 can also have depression(s) or recess(es) configured to support the one or more magnets. In any embodiments, the magnets can be disc magnets, such as neodymiun disc magnets. The magnets can have a diameter of approximately 0.25 in and have a pull strength of approximately 2 lbs each.

The stirring device 500 can have a support structure for supporting the one or more stirring elements 520, such as support member 522 which can have a generally planar top surface against which the one or more stirring elements can be supported. The support member 522 can be coupled with a base member 528 of the stirring device 500 using posts 532 and fasteners 534. The support member 522 can have openings aligned with a centerline of the stirring elements 520 to permit air flow through or against the stirring elements 520, though other embodiments do not have such openings. The one or more stirring elements can be fastened to the support member 522 using any suitable fasteners.

The stirring bar can rotate at a speed or level that is commensurate with the speed of the rotating or alternating magnetic field generated by the stirring elements, when the test container containing the stirring bar is positioned on the stirring device 500 in approximate alignment with one of the stirring elements 520. Any of the stirring bars disclosed herein can have a ridge or a pivot ring round a middle thereof. Additionally, any stirring device 500 embodiments disclosed herein can have a plurality of stationary electromagnets used to rotate a stirring bar in each test container. In any embodiments disclosed herein, the test container can be a test tube, a vial, a beaker, a tray with substance wells, an array of tubes, vials, beakers, or any other suitable or desired container for holding one or more substances.

Additionally, any of the test containers can be supported in a rack for easy transport of a plurality of the test containers. The stirring device 500 can be sized and configured to match a size, shape, and arrangement of the test containers or wells that can be supported by the rack. The stirring device 500 can also have indexing and/or alignment features used to ensure that the rack is properly aligned with the stirring device 500 when the rack is coupled with or positioned on or adjacent to the stirring device 500. Any embodiments of the stirring devices disclosed herein can be configured such that the axial centerlines of the stirring elements 520 are approximately aligned with the centerline of the test containers or substance wells supported in a rack when the rack is operably positioned on the stirring device 500. A robot arm or gripper component can be used to move any of the test containers, rack(s) of containers, or substance wells disclosed herein.

For example and without limitation, any embodiments of the stirring device 500 can be configured to support a long strip rack that holds twenty-four tubes or test containers, each of the test containers or tubes being in approximate alignment with an axial centerline of the spinning element 520. In other arrangements, any embodiments of the stirring device 500 can be configured to support a rectangular rack holding twelve or sixteen test containers. Using the controller (which can comprise software having a graphical user interface), the user can also define or “program” the system to turn on or off any location or group of locations at any desired time. This can allow for mixing times to be set and automatically controlled when the particular method calls for such limits.

In any embodiments, each of the stirring elements 520 can be independently controllable by a control system (that can comprise a computer) such that one or more of stir time, stir speed, start time for stirring, end time for stirring, and simply an on or off state of the stirring elements 520 can be independently controlled for each stirring element 520. Alternatively, in any embodiments disclosed herein, each of the stirring elements 520 can be commonly controllable by one or more controls, such as the controls shown in FIGS. 41-42, which can comprise any combination of a power switch 510, a speed controller element 512, and/or a timer element 514. In any embodiments, the speed controller element can comprise a common potentiometer that can allow a user to change the speed of the stirring elements 520, thereby allowing the stirring bars to spin faster or slower, depending on the particular parameters of the test substance and/or test being performed. In any embodiment, the timer element 514 can comprise a digital or mechanical timer.

As opposed to having just a single plate for one tube or flask, or having a surface with specific locations for a set number of flasks, the stirring device 500 embodiments disclosed herein can be daisy-chained together which can provide mixing for many tubes or just a few test containers. For example, a stirring device 500 can have twenty independently controllable stirring elements 520. A processing system 300 can have a plurality of stirring devices 500 that can be coupled together to be in communication with one another. In this arrangement, a common control system can be used to independently control each stirring element 520 of the plurality of stirring devices 500, or the control system can be used to control all of the stirring elements 520 together such that that one or more settings can be applied to any desired number of stirring elements 520.

The stirring device(s) 500 can be configured such that any number of the stirring elements 520 can be commonly controlled using software or otherwise to make the set up and control of the stirring of multiple test containers more efficient and simplified. The control system can be configured to turn any number of different stirring elements on or off, control the speed, duration, etc. of each spinning element 520. Such flexibility can allow for great integration with automation so that the system can be programmed to turn on various locations for mixing, mix for a pre-determined amount of time and turn off, then turn on other locations, etc. Further, in any embodiments, the mixing time or power on time for each spinning element 520 can be determined by a feedback mechanism wherein the reaction (along with a sensor) within the tube can determine when the test substance has reached a certain predetermined state such that mixing can end, mixing can start, mixing velocity can be reduced, and/or mixing velocity can be increased, automatically.

Any embodiments of the processing system 300 having the stirring device 500 can be automated to perform any one, or any combination of, the following activities, in any desired order:

positioning one more test containers or other containers adjacent to the top surface of the stirring device 500 so that a centerline each of such test containers is in approximate alignment with an axial centerline of a respective rotating or stirring element 520;

approximately aligning the one or more test containers with the axial centerline of one or more rotating elements 520;

initiating the stirring of one or more test containers by activating one or more stirring elements 520;

adjusting a velocity of the rotating element 520; and

terminating the stirring of one or more test containers by terminating one or more stirring elements 520.

Additionally, using the bar code reader, a specific bar code can be used to automatically trigger any one of the steps listed above or otherwise described with respect the stirring devices or any other devices disclosed herein. Additionally, any test plate processing system embodiments disclosed herein can have one or more plate readers. Further, any embodiments of the test plate processing system disclosed herein can be configured to move any processed test plate to a plate reader for further analysis.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the Figures. Depending on the embodiment, certain of the steps described above may be removed, others may be added. Accordingly, the scope of the present disclosure is intended to be defined only by reference to the claims of the utility application. The accompanying claims and their equivalents are intended to cover such forms or modifications as would fall within the scope and spirit of the protection. Furthermore, the features and attributes of the specific embodiments disclosed above may be combined in different ways to form additional embodiments, all of which fall within the scope of the present disclosure. Although the present disclosure provides certain embodiments and applications, other embodiments that are apparent to those of ordinary skill in the art, including embodiments which do not provide all of the features and advantages set forth herein, are also within the scope of this disclosure. Accordingly, the scope of the present disclosure is intended to be defined only by reference to the appended claims or claims that will be added in the future.

Accordingly, although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future. Finally, as used herein and unless otherwise stated, the term approximately is meant to represent a range of +/−10% of the stated value.

It should be emphasized that many variations and modifications may be made to the herein-described embodiments, the elements of which are to be understood as being among other acceptable examples. All such modifications and variations are intended to be included herein within the scope of this disclosure and protected by the following claims. Moreover, any of the steps described herein can be performed simultaneously or in an order different from the steps as ordered herein. Moreover, as should be apparent, the features and attributes of the specific embodiments disclosed herein may be combined in different ways to form additional embodiments, all of which fall within the scope of the present disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Moreover, the following terminology may have been used herein. The singular forms “a,” “an,” and “the” include plural referents unless the context clearly dictates otherwise. Thus, for example, reference to an item includes reference to one or more items. The term “ones” refers to one, two, or more, and generally applies to the selection of some or all of a quantity. The term “plurality” refers to two or more of an item. The term “about” or “approximately” means that quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “about 1 to 5” should be interpreted to include not only the explicitly recited values of about 1 to about 5, but should also be interpreted to also include individual values and sub-ranges within the indicated range. Thus, included in this numerical range are individual values such as 2, 3 and 4 and sub-ranges such as “about 1 to about 3,” “about 2 to about 4” and “about 3 to about 5,” “1 to 3,” “2 to 4,” “3 to 5,” etc. This same principle applies to ranges reciting only one numerical value (e.g., “greater than about 1”) and should apply regardless of the breadth of the range or the characteristics being described. A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

1. A processing device for processing one or more test plates, comprising:
a housing;
a base member coupled with the housing, the base member having a working surface against which a test plate is operably positionable,
a support member in communication with the housing and movable between a first position and a second position; and
a suction element supported directly or indirectly by the support member;
wherein:
the processing device is configured to communicate a suction force through the suction element;
the processing device is configured such that, when the support member is in the first position, an end portion of the suction element is positioned away from a working surface of the base member by a first distance, the first distance being in a direction that is normal to the working surface of the base member;
the processing device is configured such that, when the support member is in the second position, the end portion of the suction element is positioned within a second distance away from the working surface of the base member, the second distance being in a direction that is normal to the working surface of the base member; and
the second distance is significantly smaller than the first distance.

2. The processing device of claim 1, further comprising at least one opening extending through the working surface of the base member, wherein the processing device is configured to communicate a suction force through the at least one opening.

3. (canceled)

4. The processing device of claim 1, wherein the processing device is configured such that, when the support member is in the second position, the end portion of the suction element is positionable so as to be in contact with the working surface of the base member.

5. (canceled)

6. (canceled)

7. (canceled)

8. (canceled)

9. (canceled)

10. The processing device of claim 1, comprising an actuator configured to controllably move the support member between the first and second positions.

11. The processing device of claim 1, wherein the support member is coupled to a shaft of an actuator rotatable between a first angular orientation wherein the support member is in the first position and a second angular orientation wherein the support member is in the second position.

12. The processing device of claim 1, wherein a first end portion of the support member is coupled with a rotatable shaft and the suction element is coupled with a the second end portion of the support member.

13. The processing device of claim 1, wherein the suction element comprises a suction cup.

14. (canceled)

15. The processing device of claim 1, wherein the suction element is configured to rotate about an arcuate path between the first and second positions.

16. (canceled)

17. The processing device of claim 1, wherein the test plate is a 3M PETRIFILM plate.

18. The processing device of claim 1, further comprising a holder element for supporting the test plate during processing, the holder element being operably coupleable with the base member of the housing.

19. (canceled)

20. A test plate processing system, comprising the processing device of claim 1 and a source of reduced pressure configured to provide a reduced pressure to at least one of the suction elements.

21. A test plate processing system, comprising the processing device of claim 2 and a source of reduced pressure configured to provide a reduced pressure to at least one of the suction elements and the opening extending through the base member.

22. A test plate processing system, comprising:
more processing devices of claim 1;
an automated test plate storage device having one or more test plate storage modules configured to house a plurality of test plates; and
a test plate transport device configured to move one or more test plates to and from the test plate storage device, the test plate storage device being controllable using a control system.

23. The test plate processing system of claim 22, comprising two or more processing devices.

24. (canceled)

25. (canceled)

26. (canceled)

27. (canceled)

28. (canceled)

29. A test plate processing system configured to process multiple test plates, comprising:
one or more software controlled processing devices, each having a working surface, and a lifting mechanism configured to move a cover of the test plate between a first position and a second position relative to a plating surface of the test plate, wherein:
the plating surface is a surface of the test plate selectively coverable with a cover of the test plate; and
the cover is further away from the plating surface in the first position than in the second position;
a liquid handler configured to place a predetermined amount of a test substance on the plating surface of the test plate when the cover is in the first position;
a software controlled test plate storage device having one or more test plate storage modules configured to house a plurality of test plates; and
a software controlled test plate transport device configured to move one or more test plates to and from the test plate storage device.

30. The test plate processing system of claim 29, wherein the lifting mechanism of the one or more processing devices comprises a suction element.

31. (canceled)

32. (canceled)

33. (canceled)

34. (canceled)

35. (canceled)

36. (canceled)

37. (canceled)

38. A method of positioning a substance on a plating surface of a test plate having a cover using a software controlled automated processing system, comprising:
positioning the test plate in an operable position on a processing device;
selectively coupling a portion of the processing device with a portion of the cover of the test plate;
moving the cover away from the plating surface of the test plate;
dispensing a desired amount of a test substance on the plating surface of the test plate; and
moving the cover against the plating surface of the test plate.

39. The method of processing a test plate of claim 38, further comprising moving a test plate directly or indirectly from a storage device to the processing device.

40. The method of processing a test plate of claim 39, further comprising moving a test plate directly or indirectly from the processing device to the storage device.

41. (canceled)

42. The method of processing a test plate of claim 38, comprising applying suction to selectively couple the cover to a support arm of the processing device.

43. (canceled)

44. (canceled)

45. (canceled)
